SEQUENCE LISTING (56 sequences)

SEQ ID NO: 1 - DNA, 20 bp, Artificial sequence
  Synthetic oligonucleotide
ggggtcaacgttgagggggg

SEQ ID NO: 2 - DNA, 20 bp, Artificial sequence
  Synthetic oligonucleotide
gggggacgatcgtcgggggg

SEQ ID NO: 3 - DNA, 22 bp, Artificial sequence
  Synthetic oligonucleotide
gggggacgatatcgtcgggggg

SEQ ID NO: 4 - DNA, 22 bp, Artificial sequence
  Synthetic oligonucleotide
gggggacgacgtcgtcgggggg

SEQ ID NO: 5 - DNA, 22 bp, Artificial sequence
  Synthetic oligonucleotide
gggggacgagctgctcgggggg

SEQ ID NO: 6 - DNA, 20 bp, Artificial sequence
  Synthetic oligonucleotide
gggggacgtacgtcgggggg

SEQ ID NO: 7 - DNA, 21 bp, Artificial sequence
  Synthetic oligonucleotide
gggggacgatcgttggggggg

SEQ ID NO: 8 - DNA, 19 bp, Artificial sequence
  Synthetic oligonucleotide
ggggaacgatcgtcggggg

SEQ ID NO: 9 - DNA, 21 bp, Artificial sequence
  Synthetic oligonucleotide
ggggggacgatcgtcgggggg

SEQ ID NO: 10 - DNA, 21 bp, Artificial sequence
  Synthetic oligonucleotide
gggggacgatcgtcggggggg

SEQ ID NO: 11 - DNA, 21 bp, Artificial sequence
  Synthetic oligonucleotide
gggggtcatcgatgagggggg

SEQ ID NO: 12 - DNA, 20 bp, Artificial sequence
  Synthetic oligonucleotide
ggggtcgtcgacgagggggg

SEQ ID NO: 13 - DNA, 22 bp, Artificial sequence
  Synthetic oligonucleotide
ggggtcgttcgaacgagggggg

SEQ ID NO: 14 - DNA, 22 bp, Artificial sequence
  Synthetic oligonucleotide
ggggacgttcgaacgtgggggg

SEQ ID NO: 15 - DNA, 22 bp, Artificial sequence
  Synthetic oligonucleotide
ggggaacgacgtcgttgggggg

SEQ ID NO: 16 - DNA, 20 bp, Artificial sequence
  Synthetic oligonucleotide
ggggaacgtacgtcgggggg

SEQ ID NO: 17 - DNA, 24 bp, Artificial sequence
  Synthetic oligonucleotide
ggggaacgtacgtacgttgggggg

SEQ ID NO: 18 - DNA, 20 bp, Artificial sequence
  Synthetic oligonucleotide
ggggtcaccggtgagggggg

SEQ ID NO: 19 - DNA, 24 bp, Artificial sequence
  Synthetic oligonucleotide
ggggtcgacgtacgtcgagggggg

SEQ ID NO: 20 - DNA, 22 bp, Artificial sequence
  Synthetic oligonucleotide
ggggaccggtaccggtgggggg

SEQ ID NO: 21 - DNA, 19 bp, Artificial sequence
  Synthetic oligonucleotide
gggtcgacgtcgagggggg

SEQ ID NO: 22 - DNA, 18 bp, Artificial sequence
  Synthetic oligonucleotide
ggggtcgacgtcgagggg

SEQ ID NO: 23 - DNA, 22 bp, Artificial sequence
  Synthetic oligonucleotide
ggggaacgttaacgttgggggg

SEQ ID NO: 24 - DNA, 19 bp, Artificial sequence
  Synthetic oligonucleotide
ggggacgtcgacgtggggg

SEQ ID NO: 25 - DNA, 20 bp, Artificial sequence
  Synthetic oligonucleotide
gggggtcgttcgttgggggg

SEQ ID NO: 26 - DNA, 19 bp, Artificial sequence
  Synthetic oligonucleotide
gggacgatcgtcggggggg

SEQ ID NO: 27 - DNA, 20 bp, Artificial sequence
  Synthetic oligonucleotide
gggtcgtcgacgaggggggg

SEQ ID NO: 28 - DNA, 19 bp, Artificial sequence
  Synthetic oligonucleotide
ggtcgtcgacgaggggggg

SEQ ID NO: 29 - DNA, 20 bp, Artificial sequence
  Synthetic oligonucleotide
ggggacgatcgtcggggggg

SEQ ID NO: 30 - DNA, 27 bp, Artificial sequence
  Synthetic oligonucleotide
ggggtcgacgtcgacgtcgaggggggg

SEQ ID NO: 31 - DNA, 21 bp, Artificial sequence
  Synthetic oligonucleotide
ggggacgacgtcgtggggggg

SEQ ID NO: 32 - DNA, 30 bp, Artificial sequence
  Synthetic oligonucleotide
gggggggggggacgatcgtcgggggggggg

SEQ ID NO: 33 - DNA, 22 bp, Artificial sequence
  Synthetic oligonucleotide
tcgtcgttttcggcgcgcgccg

SEQ ID NO: 34 - DNA, 22 bp, Artificial sequence
  Synthetic oligonucleotide
tcgtcgttttcggcggccgccg

SEQ ID NO: 35 - DNA, 22 bp, Artificial sequence
  Synthetic oligonucleotide
tcgtcgttttcggcgcgccgcg

SEQ ID NO: 36 - DNA, 22 bp, Artificial sequence
  Synthetic oligonucleotide
tcgtcgttttcggcgccggccg

SEQ ID NO: 37 - DNA, 22 bp, Artificial sequence
  Synthetic oligonucleotide
tcgtcgttttcggcccgcgcgg

SEQ ID NO: 38 - DNA, 27 bp, Artificial sequence
  Synthetic oligonucleotide
tcgtcgttttcggcgcgcgccgttttt

SEQ ID NO: 39 - DNA, 22 bp, Artificial sequence
  Synthetic oligonucleotide
tcctgacgttcggcgcgcgccg

SEQ ID NO: 40 - DNA, 22 bp, Artificial sequence
  Synthetic oligonucleotide
  modified_base (2)..(2): m5c
  modified_base (5)..(5): m5c
  modified_base (11)..(11): m5c
  modified_base (14)..(14): m5c
  modified_base (16)..(16): m5c
  modified_base (18)..(18): m5c
  modified_base (20)..(21): m5c
tcgtcgttttcggcgcgcgccg

SEQ ID NO: 41 - DNA, 22 bp, Artificial sequence
  Synthetic oligonucleotide
tcctgacgttcggcgcgcgccc

SEQ ID NO: 42 - DNA, 22 bp, Artificial sequence
  Synthetic oligonucleotide
tcggcgcgcgccgtcgtcgttt

SEQ ID NO: 43 - DNA, 22 bp, Artificial sequence
  Synthetic oligonucleotide
tcgtcgttttcggcggccgacg

SEQ ID NO: 44 - DNA, 22 bp, Artificial sequence
  Synthetic oligonucleotide
tcgtcgttttcgtcggccgccg

SEQ ID NO: 45 - DNA, 22 bp, Artificial sequence
  Synthetic oligonucleotide
tcgtcgttttcgacggccgccg

SEQ ID NO: 46 - DNA, 22 bp, Artificial sequence
  Synthetic oligonucleotide
tcgtcgttttcggcggccgtcg

SEQ ID NO: 47 - DNA, 21 bp, Artificial sequence
  Synthetic oligonucleotide
tcgtcgtttcgacggccgtcg

SEQ ID NO: 48 - DNA, 21 bp, Artificial sequence
  Synthetic oligonucleotide
tcgtcgtttcgacgatcgtcg

SEQ ID NO: 49 - DNA, 21 bp, Artificial sequence
  Synthetic oligonucleotide
tcgtcgtttcgacgtacgtcg

SEQ ID NO: 50 - DNA, 18 bp, Artificial sequence
  Synthetic oligonucleotide
tcgtcgcgacggccgtcg

SEQ ID NO: 51 - DNA, 18 bp, Artificial sequence
  Synthetic oligonucleotide
tcgtcgcgacgatcgtcg

SEQ ID NO: 52 - DNA, 18 bp, Artificial sequence
  Synthetic oligonucleotide
tcgtcgcgacgtacgtcg

SEQ ID NO: 53 - DNA, 22 bp, Artificial sequence
  Synthetic oligonucleotide
tcgtttttttcgacggccgtcg

SEQ ID NO: 54 - DNA, 22 bp, Artificial sequence
  Synthetic oligonucleotide
tcgtttttttcgacgatcgtcg

SEQ ID NO: 55 - DNA, 22 bp, Artificial sequence
  Synthetic oligonucleotide
tcgtttttttcgacgtacgtcg

SEQ ID NO: 56 - DNA, 116 bp, Artificial sequence
  Synthetic oligonucleotide
aggtggtaacccctaggggttaccaccttcattggaaaacgttcttcggggcgttcttaggtggtaacccctaggggttaccaccttcattggaaaacgttcttcggggcgttctt

(19) United States
(12) Patent Application Publication
(10) Pub. No.: US 2019/0046638 A1
(43) Pub. Date: Feb. 14, 2019

(54) Fc RECEPTOR-MEDIATED DRUG DELIVERY

(71) Applicant: CHECKMATE PHARMACEUTICALS, INC., Cambridge, MA (US)

(72) Inventor: Arthur M. KRIEG, Cambridge, MA (US)

(21) Appl. No.: 16/089,675

(22) PCT Filed: Mar. 31, 2017
(86) PCT No.: PCT/US2017/025480

§ 371 Date: Sep. 28, 2018

Related U.S. Application Data
(60) Provisional application No. 62/316,674, filed on Apr. 1, 2016.

(51) Int. Cl. A61K 39/39; A61P 37/04

(52) U.S. Cl. CPC A61K 39/39; A61K 2039/55561; A61P 37/04

(57) ABSTRACT

Provided are methods and compositions for modulating an immune response or for treating a disease or condition in a subject, such as cancer, infection, autoimmune disease, allergy, and asthma. The methods involve systemically administering to a subject a particle comprising a surface and an interior, wherein the surface of the particle comprises an antigen, the interior of the particle comprises an immune modulating agent, and the subject is or has been primed to mount an antibody response to the antigen. The antibody response to the particle permits Fc receptor-bearing target cells to take up the particle, thereby delivering the immune modulating agent to the target cells and modulating an immune response of the subject. In various embodiments, the immune modulating agent can be selected from the group consisting of therapeutic agents, immune activators, and immune suppressors. In certain embodiments, the immune activator is a TLR agonist, e.g, a CpG oligodeoxynucleotide.

### RELATED APPLICATIONS

This application claims benefit of U.S. Provisional Patent Application No. 62/316,674, filed Apr. 1, 2016.

### SEQUENCE LISTING

The instant application contains a Sequence Listing which has been submitted electronically in ASCII format and is hereby incorporated by reference in its entirety. Said ASCII copy, created on Mar. 31, 2017, is named 589637_CPS-002PC_ST25 and is 11,833 bytes in size.

### BACKGROUND OF THE INVENTION

The targeted delivery of drugs into specific cells or tissues has tremendous potential for therapeutic benefit, and therefore has been a focus of attention for drug developers for many years. The state of the art has produced many major advances in targeted delivery, using a wide range of nanoparticles, dendrimers, etc., which can be targeted using a diverse range of ligands, including for example antibodies, antibody fragments such as Fc, or via the neonatal FcR. However, these are generally quite complicated to manufacture, and inclusion of all of the components required frequently results in the generation of a particle, e.g., a negatively charged particle, that induces complement activation, reducing the therapeutic index of any drug.

Mammalian receptors for the Fc domain or region of immunoglobulins (FcR) are transmembrane molecules (reviewed by Van de Winkel and Capel (1993) Immunol. Today 14:215). These receptors provide a feedback between the humoral and cellular immune responses. Their interaction with immunoglobulins triggers immune functions such as phagocytosis, cytotoxicity, cytokine release, and enhancement of antigen presentation.

FcR are members of the immunoglobulin superfamily, and they include various different receptors for the Fc domain of various different types of immunoglobulins (IgG, IgE, IgA, and IgM). In humans several classes of receptors for the Fc domain of IgG (FcγR) are recognized, including hFcγRI (CD64), hFcγRIIA (CD32), hFcγRIIB1 (CD32), hFcγRIIB2 (CD32), hFcγRIIIA (CD16a), hFcγRIIIB (CD16b), and FcRn. hFcγRI is unique in its capacity to bind with high affinity to monomeric IgG (Ka=10⁸-10⁹ M⁻¹). Its binding is strong to human IgG3, IgG1, and IgG4 (with decreasing affinity), and to mouse IgG2a and IgG3, whereas binding to human IgG2 and mouse IgG1 and IgG2b is much weaker. hFcγRI is constitutively expressed on monocytes and macrophages, and its expression can be induced on neutrophils and eosinophils.

Certain FcγR are stimulatory, and certain other FcγR are inhibitory. For example, certain FcγR generate signals within their cells through an activation motif known as an immunoreceptor tyrosine-based activation motif (ITAM). An ITAM is a specific sequence of amino acids (YXXL) occurring twice in close succession in the intracellular tail of a receptor. When phosphate groups are added to the tyrosine (Y) residue of the ITAM by tyrosine kinases, a signaling cascade is generated within the cell. This phosphorylation reaction typically follows interaction of an Fc receptor with its ligand. An ITAM is present in the intracellular tail of FcγRIIA, and its phosphorylation induces phagocytosis in macrophages. FcγRI and FcγRIIIA do not have an ITAM but can transmit an activating signal to their phagocytes by interacting with another protein that does. This adaptor protein is called the Fcγ subunit and, like FcγRIIA, contains the two YXXL sequences that are characteristic of an ITAM.

The presence of only one YXXL motif is not sufficient to activate cells and represents a motif (S/I/V/LXYXXI/V/L) known as an immunoreceptor tyrosine-based inhibitory motif (ITIM). FcγRIIB1 and FcγRIIB2 have an ITIM sequence and are inhibitory Fc receptors; they do not induce phagocytosis. Inhibitory actions of these receptors are controlled by enzymes that remove phosphate groups from tyrosine residues. The phosphatases SHP-1 and SHIP-1 inhibit signaling by Fcγ receptors.

Plasmacytoid dendritic cells (pDCs) are innate immune cells that circulate in the blood and are found in peripheral lymphoid organs. In addition, pDC are one of the types of DC present in the liver (Lukacs-Kornek, V. et al. (2013) “Dendritic cells in liver injury and fibrosis: shortcomings and promises.” J Hepatol 59(5):1124-6) and in tumors generally, where immature tumor-infiltrating pDC have been shown to promote immune tolerance to the tumor, facilitating tumor growth (Lombardi, V. C. et al. (2015) “Plasmacytoid dendritic cells, a role in neoplastic prevention and progression.” Eur J Clin Invest 45:1-8). They constitute <0.4% of peripheral blood mononuclear cells (PBMC). In humans these cells express the surface markers CD123, BDCA-2 (CD303) and BDCA-4 (CD304), but do not express high levels of CD11c or CD14, which distinguishes them from conventional dendritic cells or monocytes, respectively. In addition, pDC express the activating FcγRIIA, enabling them to take up and be activated by immune complexes. Mathan, T. S. et al. (2013) “Human plasmacytoid dendritic cells: from molecules to intercellular communication network.” Front Immunol 4:372. As components of the innate immune system, these cells express intracellular Toll-like receptors TLR7 and TLR9 which detect single-stranded RNA and unmethylated CpG DNA sequences, respectively, within an endosomal compartment. pDC can take up and be activated by isolated TLR7 or TLR9 ligands, or by TLR7/9 ligands in the form of immune complexes, which are internalized through FcγRIIA. Upon stimulation and subsequent activation through TLR7 or TLR9, these cells produce large amounts of type I interferon (mainly the various IFN-α isoforms and IFN-β) which are critical pleiotropic anti-viral compounds mediating a wide range of effects, and may become able to mediate tumor regression, instead of growth. Lombardi, V. C. et al. (2015) Eur J Clin Invest 45:1-8.

Type I interferons are a subgroup of interferons and include 13 isotypes of IFN-α, IFN-β, IFN-κ, IFN-δ, IFN-ε, and IFN-ω which can be secreted by many cell types including lymphocytes (T cells, B cells, and natural killer (NK) cells), macrophages, fibroblasts, endothelial cells, osteoblasts and others. They stimulate both macrophages and NK cells to elicit an anti-viral response, and are also active against tumors. Plasmacytoid dendritic cells have been identified as being by far the major producers of type I IFNs in response to infection, and have thus been coined natural IFN producing cells.

### SUMMARY OF THE INVENTION

The present invention provides delivery vehicles for delivering drugs and other agents to a target cell that expresses an FcR. The delivery vehicles of the invention are characterized as particles which are constructed and arranged so as to be capable of inducing or exploiting an antibody response by a subject. Antibodies then bind to the particles, facilitating their uptake by Fc receptors expressed on target cells. Notably, the delivery vehicle itself does not include an Fc domain; rather, upon administration to a subject, the delivery vehicle evokes or uses an immune response which results in attachment, in vivo, of antibodies directed against a component of the vehicle. The resulting opsonized particles can be taken up by Fc receptor-bearing target cells.

The present invention also provides methods using particles of the invention to deliver a drug or other payload to an FcR-expressing cell; to modulate an immune response in a subject; and to treat a disease or condition of a subject.

The particles and methods of the invention are useful in the treatment of various diseases and conditions, including, in particular, certain infections, cancers, and autoimmune and inflammatory diseases. The particles and methods of the invention are particularly useful in the treatment of viral hepatitis and primary and metastatic liver cancer.

Particles in accordance with the invention comprise a surface and an interior, wherein the surface of the particle comprises an antigen capable of being bound by an antibody that binds specifically to the antigen, and the interior of the particle optionally comprises a payload compound. Generally, particles with a payload can be used either to immunize or to treat a subject, and particles without a payload can be used to immunize a subject.

Following one or more priming doses of the antigen or particle through an immunogenic route of administration, one or more therapeutic doses of the particle are administered through a relatively non-immunogenic route of administration, e.g., intravenously, in order to deliver the payload compound more broadly through the body, and in particular to the liver and other reticuloendothelial system (RES) tissues.

In certain embodiments, a subject's immune system has already been primed to respond to the antigen, or to a molecular mimic of the antigen, and no additional priming dose is required. In certain other embodiments, a subject's immune system has not already been primed to respond to the antigen, and one or more priming doses are required. In yet certain other embodiments, a subject's immune system has already been primed to respond to the antigen, and one or more boosting doses are administered to the subject.

An aspect of the invention is a method of modulating an immune response, comprising:

systemically administering to a subject in need thereof an effective amount of a particle comprising a surface and an interior, wherein the surface of the particle comprises an antigen, the interior of the particle comprises an immune modulating agent, and the subject is primed to mount an antibody response to the antigen, to modulate an immune response of the subject.

An aspect of the invention is a method of modulating an immune response, comprising:

immunogenically administering to a subject in need thereof an effective amount of a first particle comprising a surface and an interior, wherein the surface of the first particle comprises an antigen, and the interior of the first particle optionally comprises a first immune modulating agent, to immunize the subject against the antigen; and

systemically administering to the subject an effective amount of a second particle comprising a surface and an interior, wherein the surface of the second particle comprises the antigen, and the interior of the second particle comprises a second immune modulating agent, to modulate an immune response of the subject.

The priming or immunizing step involves administration via an immunogenic route, including, e.g., subcutaneous (SC), intramuscular (IM), intradermal (ID), transdermal, or mucosal, but normally is not intravenous (IV) because IV administration typically is tolerizing, or not immunogenic, and the purpose of the priming step is to induce the synthesis of antibodies to the antigen, which requires an immunogenic route of administration. This same principle also applies to boosting doses.

An aspect of the invention is a method of treating a disease or condition, comprising:

systemically administering to a subject having a disease or condition an effective amount of a particle comprising a surface and an interior, wherein the surface of the particle comprises an antigen, the interior of the particle comprises an immune modulating agent, and the subject is primed to mount an antibody response to the antigen, to modulate an immune response of the subject,

thereby treating the disease or condition.

An aspect of the invention is a method of treating a disease or condition, comprising:

immunogenically administering to a subject having a disease or condition an effective amount of a first particle comprising a surface and an interior, wherein the surface of the first particle comprises an antigen, and the interior of the first particle optionally comprises a first immune modulating agent, to immunize the subject against the antigen; and

systemically administering to the subject an effective amount of a second particle comprising a surface and an interior, wherein the surface of the second particle comprises the antigen, and the interior of the second particle comprises a second immune modulating agent, to modulate an immune response of the subject,

thereby treating the disease or condition.

### BRIEF DESCRIPTION OF THE DRAWINGS

FIG. 1 is a pair of graphs depicting serum concentrations of chemokines and cytokines upregulated in vivo in mice as measured 3 hours following intravenous (IV) bolus dosing of the indicated amounts of CMP-001.

FIG. 2A is a series of in vivo images depicting dorsal views of mice taken at the indicated times following IV administration of fluorescently labeled CMP-001. Original images are in color.

FIG. 2B is a series of in vivo images depicting ventral views of mice taken at the indicated times following IV administration of fluorescently labeled CMP-001. Original images are in color.

FIG. 2C is a graph depicting total radiant efficiency of liver region of mice taken at the indicated times following IV administration of fluorescently labeled CMP-001. Each point represents mean±SEM for three mice.

### DETAILED DESCRIPTION OF THE INVENTION

Particularly for cancer therapy or chronic liver infections such as HBV, HCV, or HIV, there has been a great interest in ways to improve the targeting of therapeutics to the liver. An improved liver-targeted delivery system could be useful for the treatment of either primary liver cancer (hepatocellular carcinoma, HCC) or cancer that is metastatic to the liver, which is a major source of mortality and morbidity. Many nanoparticles have been designed to target the liver hepatocytes, especially such as through GalNac conjugation (see, for example, U.S. Pat. No. 8,450,467 to Alnylam). However, none of these have been designed to deliver the payload via an FcR, unless the particle as provided comprises an Fc domain Prior to the present invention, there was no particle known in the art that provides a method for selectively activating a dendritic cell (DC), or a particular type of DC such as a plasmacytoid DC (pDC), by inducing the formation of circulating immune complexes (CIC) that deliver a therapeutic agent via an FcR into the DC or pDC. There was no therapeutic method described that uses such a delivery method for a particle to deliver an agonist for TLR7 or TLR9 that will selectively activate pDC without activating other immune cell types, thereby providing an improved safety profile compared to therapeutics known in the art that activate diverse immune cell types. By selectively activating only pDC, and not monocytes, macrophages, and other FcR-expressing cells, a therapeutic particle can provide an enormously improved safety profile compared to the immune stimulatory compounds or formulations known in the art, that activate a greater variety of immune cell types. The IV route of administration provides a method for particles of the invention to activate liver pDC, inducing the production of type I IFN in the liver and promoting the generation and influx of antigen-specific CD4⁺ and CD8⁺ T cells for the treatment of liver infections (e.g., HBV, HCV, HIV) or cancer, including primary hepatocellular cancer (HCC), as well as cancer metastatic to the liver. Conversely, particles of the invention containing TLR7 and/or TLR9 (TLR7/9) antagonists can be used in the treatment of diseases characterized by inappropriate and/or undesirable pDC activation, such as systemic lupus erythematosus (SLE), psoriasis, rheumatoid arthritis (RA), nonalcoholic steatohepatitis (Garcia-Martinez, I. et al. (2016) “Hepatocyte mitochondrial DNA drives nonalcoholic steatohepatitis by activation of TLR9.” J Clin Invest 126.3), and other autoimmune and inflammatory diseases. Particles containing agonists or antagonists for innate immune receptors such as TLRs, RLRs, NLRs, and etc. that are expressed in other subsets of FcR-expressing cells can likewise be used in the treatment of diseases characterized by a positive or negative defect in the function of such other FcR-expressing cells based on the published expression patterns of these receptors. Percutaneous or intra-hepatic arterial injection of therapeutics has been proposed but is invasive and carries substantial morbidity, as well as a non-negligible risk of mortality.

The particles of the invention have the advantage over previous particles designed to deliver drugs into an FcR-expressing cell of being much simpler to manufacture, since the particles do not actually have to be produced with an Fc domain already on the particle. Instead, following a priming dose through an immunogenic route of administration, e.g., subcutaneous injection, of the particle, the subject's immune system automatically generates an antibody response to the particles, such that upon a subsequent systemic (e.g., intravenous) administration of the particles (or of antigenically-related particles), the subject's antibodies to the surface antigen coat (opsonize) the particles, exposing the Fc domain of the antibody, and resulting in the particle's systemic uptake by FcR-expressing cells in the liver and elsewhere. Although therapeutic methods of administering immunogenic particles through either an immunogenic route (such as subcutaneous) or a non-immunogenic route (such as intravenous) are well described in the art, there is no prior description of an approach that is based on the administration of a priming immunogenic dose of a particle followed by a systemic administration of the particle (or of an antigenically-related particle) for the purpose of forming immune complexes of the particle in the circulation in order to deliver a therapeutic payload into FcR-expressing cells.

At a molecular level, in certain embodiments the invention comprises a particle that contains an antigen on the surface, and an adjuvant; the adjuvant can be present on the surface or as a payload. The adjuvant or the antigen may be a therapeutic, or the particle optionally comprises an additional therapeutic (as a third component of the particle) or therapeutics, or diagnostic or imaging agents. The therapeutic may be a small molecule (e.g., chemotherapy; anti-angiogenic; poly-ADP ribose polymerase (PARP) inhibitor; a cancer therapeutic; a protein (peptide, “stapled peptide”, toxic peptide, protein to intracellular antigen); or a nucleic acid, either as an innate immune activator, an aptamer, exon-skipping or splice-modulating oligonucleotide, antisense, RNAi, micro-RNA targeting, or mRNA therapeutic (e.g., encoding a therapeutic or a vaccine antigen, including an antigen to which tolerance is desired to be induced). In the case of cancer immunotherapy, a preferred adjuvant is an agonist for TLR7 and/or TLR9; particularly preferred are adjuvants that induce a very high induction of type I IFN (by activating IRF7) with little or no significant activation of NF-κB, such as CpG-A or the 3M small-molecule TLR7 and TLR7/8 agonists such as the imidazoquinolines as are well known in the art.

At a cellular level, the surface antigen can be bound by antibodies (opsonized) and the particle then can be internalized by any FcR-expressing cell, such as a monocyte, macrophage, dendritic cell, pDC, B cell, natural killer (NK) cell, neutrophil, eosinophil, or mast cell. Phagocytic cells as well as B cells and conventional dendritic cells express the inhibitory FcR, FcγRIIB, by which particle uptake inhibits other immune responses. However, pDC do not express the inhibitory FcR, only the stimulatory FcR, FcγRIIA, and therefore the uptake of an opsonized particle by a pDC has a fundamentally different immune effect from the uptake of such a particle by any other immune cell subset: there is the potential for synergy with signaling induced by any TLR7 or TLR9 agonist within the particle. Since human pDC uniquely express only TLR7 and TLR9, other payloads within the particle may not have this particular effect. Basophils express only inhibitory FcR, and no stimulatory FcR, and so the invention can be used to deliver an “off” signal to basophils, or a therapeutic that acts selectively only on basophils.

Phagocytic cells will take up the particles into an endosomal compartment in which they are digested, thereby potentially releasing the therapeutic/imaging agent, which may be active either in the cell which took up the agent, or in other cells in the tissue. Depending on the contents of the particle, the phagocytic cells may be activated or inhibited or not affected at all by the adjuvant and/or therapeutic moiety. In the case of cells that are not “professional phagocytes”, such as DC or B cells, the particles also are “scanned” in the endosomes (or in some cases, by cytoplasmic receptors) for the presence of pathogen-associated molecular patterns (PAMPS), which activate innate immunity through pathways such as the TLRs, nucleotide-binding oligomerization domain receptors (NLRs), RIG-I-like receptors (RLRs), and mitochondrial antiviral-signaling protein (MAVS). See, for example, Sharma S. et al. (2015) “Nucleic acid-sensing receptors: rheostats of autoimmunity and autoinflammation.” J Immunol 195(8):3507-12; Kawai T. et al. (2011) “Toll-like receptors and their crosstalk with other innate receptors in infection and immunity.” Immunity 34(5):637-50. In addition to FcR-mediated uptake through their inhibitory FcR, which normally would provide an “off” signal to a B cell internalizing the particles of the present invention, B cells expressing a B cell antigen receptor that binds to any surface antigen of the particle (including antigens that formed after the administration of the particle to the subject) will take up the particles via the B cell receptor, through which synergy occurs if the cell is exposed to a TLR7 or TLR9 agonist at the same time (such as within the particle).

At a tissue level, any opsonized particles will be taken up directly by FcR-bearing cells but not directly by cells in the tissue which do not express FcR, unless the particle has been specifically designed for such uptake. For example, unless the particle design is optimized for uptake into hepatocytes (such as with lipid nanoparticles used for RNAi administration), then hepatocytes generally will not be targeted, and the opsonized particles instead will be taken up by Kupffer cells and other liver DC expressing FcR, including liver pDC (for a review of liver DC, see Lukacs-Kornek et al. (2013) J Hepatology 59:1124-6). Within those cell types the particles can be degraded to release a therapeutic agent that will be active in the tissue as a whole (e.g., a chemotherapeutic agent in a liver cancer). Alternatively or in addition, particles that contain a highly type I IFN-inducing TLR7/9 ligand can activate pDC within the tissue, even reversing pDC suppression in the settings of liver cancer or chronic viral infections. Such pDC activation can dramatically change the immune state of a tissue, converting immunologically “cold” tissues into “hot” ones, in which high levels of type I IFN production promote the activation of other immune cells, including CD8⁺ T cells, NK cells, and other dendritic cell populations.

At an organismal level, the invention in certain embodiments involves two steps, including a “priming dose” and a “therapeutic dose”. The purpose of the “priming dose” is to induce an antibody (Ab) response to a surface antigen of the particle. The priming dose may be administered by any of the many vaccination routes known in the art, including, e.g., injecting the particle SC, ID, or IM, by transdermal administration (or electroporation), oral, nasal, mucosal, rectal, vaginal, etc. Particularly when the particle contains an adjuvant to an antibody response, this single “priming dose” functions as a vaccine and induces an IgG antibody response to the particle antigen within a week or even less (if the adjuvant is a preferred highly active one, such as a TLR7/9 agonist). For particles comprising weaker adjuvants, a longer time or several priming doses may be required. The particle used in the priming step does not have to include a therapeutic payload, but must comprise the antigen and optionally the adjuvant. Once the Ab response has been induced, the subject is then administered the second dose (the “therapeutic dose”) of the same (or a different but antigenically related) particle, which contains the therapeutic payload. For the treatment of a liver disease (such as cancer or chronic infection) or for achieving the broadest possible systemic induction of type I IFN production by pDC, this second dose may be given systemically, e.g., IV. Such IV administration of the particles in a previously primed subject is expected to result in the formation of circulating immune complexes (CIC) as a result of the binding of the circulating anti-antigen antibodies to the antigen on the particles. Most of these CIC will be taken up by phagocytic cells (expressing high-affinity FcR) that may release the payload by degrading the particle in a phagosome. Alternatively the phagocytic cell may have no response to the payload (such as the situation when the payload is a TLR7 agonist or a TLR9 agonist, because “professional” phagocytic cells do not express these innate immune receptors). If the IV dose is high enough or is infused over a long enough time, then the rapid uptake by phagocytic cells can be saturated to a great enough degree that pDC also are able to take up the CIC. In such a case if the CIC contain a TLR7 agonist or TLR9 agonist, then the pDC are induced to secrete type I IFN and to change from an immune-tolerizing phenotype to an immune-stimulating phenotype.

At a therapeutic level, the invention provides a novel approach to the targeted delivery of a payload, be it a drug, imaging agent, or other therapeutic agent, into the tissues of the reticuloendothelial system (RES) (e.g., liver, BM), and into DC (including cDC and pDC), B cells, and other FcR-expressing cell types. In certain embodiments of the invention (in which particles contain a TLR7 agonist or TLR9 agonist, which may be either a nucleic acid or small molecule), the invention can be used for a cancer immunotherapy to markedly activate pDC throughout the body, including within tumors. Depending on the specific TLR agonist (e.g., CpG-A most preferred but RNA or small molecule TLR7 activators also can be used), this will result in a profound induction of type I IFN and can alter the tumor microenvironment in a cancer patient, potentiating the effects of other therapeutics, including cancer therapeutics and especially cancer immunotherapeutics such as checkpoint inhibitors or compounds that promote T-cell activation or chimeric antigen receptor (CAR) T cells or tumor-infiltrating lymphocyte (TIL) therapies; or in the setting of a chronic infection the IFN-α induction can promote the efficacy of other anti-viral therapeutics.

Alternatively in accordance with this invention, particles that contain a TLR7 and/or a TLR9 antagonist known in the art (such as those described in U.S. Pat. Nos. 9,126,996 and 7,410,975 and U.S. Patent Application Publication No. 2004/0009949, the entire contents of each of which are incorporated herein by reference) can be delivered into pDC by IV administration for the treatment of autoimmune diseases resulting from immune complexes containing nucleic acids that stimulate pDC, including for example, systemic lupus erythematosus (SLE), rheumatoid arthritis (RA), psoriasis, dermatomyositis, and other inflammatory and immune-mediated diseases. For reviews, see Sharma, S. et al. (2015) “Nucleic acid—sensing receptors: Rheostats of autoimmunity and autoinflammation.” J Immunol 195(8):3507-12; U.S. Pat. Nos. 9,126,996 and 7,410,975; and U.S. Patent Application Publication No. 2004/0009949.

The invention and development of immune stimulatory CpG oligodeoxynucleotides (ODN) and subsequent invention and development of various classes and designs of CpG ODN provided new opportunities for cancer immunotherapy. Based on encouraging preclinical data in rodent models, human clinical trials of CpG ODN have been performed in oncology patients using systemic and intratumoral administration of several different CpG ODN alone or in combination with various chemotherapy regimens, vaccines, antibodies, and radiotherapy. Clinical responses in these trials have been uncommon, however, and despite some encouraging early clinical trial results, phase 3 trials have so far failed (reviewed in Krieg, A. M. (2012) Nucleic Acid Ther 22(2):77-89). Therefore, there exists a need to provide improved oligonucleotide therapeutic approaches to increase the success rate of cancer immunotherapy.

CpG ODN bind and stimulate TLR9, an innate immune receptor which is constitutively expressed in only two types of human immune cell: B cells, which respond to TLR9 stimulation by proliferating and secreting immunoglobulin; and plasmacytoid dendritic cells (pDC), which respond to TLR9 stimulation by secreting large amounts of type I IFN (IFN-α and IFN-β). The present invention is based, at least in part, on the finding that the IFN-α response to CpG ODN is important for tumor immunotherapy. The present invention is based, at least in part, on the finding that a strong IFN-α response to CpG ODN is important for tumor immunotherapy, including tumor immunotherapy using intratumoral administration of CpG ODN.

Preferred CpG ODN of the invention are characterized, at least in part, by their propensity to induce high amounts of type I IFN.

Type I IFN are believed to play a key role in tumor rejection. For example, type I IFN augment CD8⁺ T-cell survival, expansion, and effector differentiation; promote dendritic cell (DC) maturation, cross-presentation of tumor-associated antigens to CD8⁺ T cells; are required for immune surveillance against carcinogen-induced tumors; and are required for rejection of implanted tumors. Additionally, recent studies have demonstrated that levels of type I IFN-related mRNA correlate with tumor-infiltrating lymphocytes (TILs) in human metastases.

In addition to inducing higher levels of type I IFN than anything else yet identified, TLR9 ligands such as CpG-A ODN also activate pDC and induce secretion of hundreds of other Th1-promoting genes and factors; and convert pDC from immature/tolerance-promoting phenotype to mature, activated, cytotoxic T lymphocyte (CTL)-inducing phenotype.

### Particles of the Invention

An aspect of the invention is a particle comprising a surface and an interior, wherein the surface of the particle comprises an antigen capable of being bound by an antibody that binds specifically to the antigen, and the interior of the particle optionally comprises a payload compound. In certain embodiments, the particle is a non-naturally occurring particle comprising a surface and an interior, wherein the surface of the particle comprises an antigen capable of being bound by an antibody that binds specifically to the antigen, and the interior of the particle optionally comprises a payload compound.

In certain embodiments, the particle is selected from the group consisting of liposomes, virus-like particles, and lipid nanoparticles. Methods for preparing such types of particles, in general, are well known in the art.

For example, methods of producing particles of the invention and loading them with nucleic acids and other agents are well known in the art, such as U.S. Pat. Nos. 8,691,209, 9,139,554, and 9,220,683, the entire contents of each of which are incorporated herein by reference. Loading of the payload is typically performed during the particle manufacture, often via subunit and solvent mixing or evaporation procedures that are well known in the art. Production of a virus-like particle using a viral or bacteriophage coat protein can take advantage of the protein properties and propensity for packaging nucleic acids for particularly efficient packaging of a nucleic acid payload, which may comprise natural or modified RNA, DNA, or other nucleic acids. Nucleic acid modifications may be to the backbone, the sugars, or the bases, as are well known in the art of nucleic acid therapeutics. The loading efficiency of the particles depends on the specific characteristics of the particle and the payload, and can vary widely, from less than 5% of the mass of the particle comprising the payload, to approximately 20% or more.

The particle generally is roughly spherical in shape but it may take other shapes including, for example, polyhedrons, oblates, cylinders, boxes, cubes, cuboids, pyramids, and irregular three-dimensional shapes.

The particle generally is about 10 nm to about 2000 nm in its greatest diameter. In some embodiments the second particle used for therapeutic delivery is larger than the first immunogenic particle used for priming. In certain embodiments, the particle is about 10 nm to about 100 nm in its greatest diameter. In certain embodiments, the particle is about 10 nm to about 200 nm in its greatest diameter. In certain embodiments, the particle is about 10 nm to about 300 nm in its greatest diameter. In certain embodiments, the particle is about 10 nm to about 400 nm in its greatest diameter. In certain embodiments, the particle is about 10 nm to about 500 nm in its greatest diameter. In certain embodiments, the particle is about 10 nm to about 1000 nm in its greatest diameter. In certain embodiments, the particle is about 100 nm to about 200 nm in its greatest diameter. In certain embodiments, the particle is about 100 nm to about 300 nm in its greatest diameter. In certain embodiments, the particle is about 100 nm to about 400 nm in its greatest diameter. In certain embodiments, the particle is about 100 nm to about 500 nm in its greatest diameter. In certain embodiments, the particle is about 100 nm to about 1000 nm in its greatest diameter. In certain embodiments, the particle is about 100 nm to about 2000 nm in its greatest diameter. In certain embodiments, the particle is about 200 nm to about 500 nm in its greatest diameter. In certain embodiments, the particle is about 200 nm to about 1000 nm in its greatest diameter. In certain embodiments, the particle is about 200 nm to about 2000 nm in its greatest diameter. In certain embodiments, the particle is about 500 nm to about 1000 nm in its greatest diameter. In certain embodiments, the particle is about 500 nm to about 2000 nm in its greatest diameter. In certain embodiments, the particle is about 1000 nm to about 2000 nm in its greatest diameter.

In general, it is believed that particles used for priming an antibody response are up to about 200 nm in greatest diameter. For example, in certain particularly preferred embodiments, the particle is about 10 nm to about 100 nm in its greatest diameter.

Also in general, a particle used for delivery of a therapeutic agent may be larger than a particle used for priming an antibody response, so that particles used for delivery of a therapeutic agent may be greater than or equal to about 200 nm in greatest diameter. For example, in certain particularly preferred embodiments, the particle is about 200 nm to about 500 nm in its greatest diameter.

The surface of the particle can be comprised of any biocompatible material, and, apart from the antigen, it can be homogeneous or heterogeneous in its composition. At least a portion of the antigen will be exposed to the environment on the outer aspect or outer face of the surface. For purposes of immunizing a subject against the antigen, at least that portion of the antigen sufficient to allow the immune system of the subject to recognize the antigen will be exposed to the environment on the outer aspect or outer face of the surface. For purposes of delivering a payload to a primed subject, at least that portion of the antigen sufficient to allow an antigen-specific antibody to bind to the antigen will be exposed to the environment on the outer aspect or outer face of the surface.

An “antigen” as used herein refers to any substance that stimulates an antibody response specific for said substance when introduced into the body. Antigens in general can be any type of molecule but typically are comprised of peptides, proteins, glycoproteins, carbohydrates, lipids, and any combination thereof.

The antigen can be naturally occurring or non-naturally occurring. In certain embodiments, the antigen is selected from the group consisting of viral antigens (including bacteriophage), bacterial antigens, and tumor antigens, including tumor neoantigens. In certain embodiments, the antigen is a viral antigen. In certain embodiments, the antigen is a bacterial antigen. In certain embodiments, the antigen is a tumor antigen. In certain embodiments, the antigen is a tumor neoantigen. Examples of each these types of antigen are well known in the art.

The antigen forms or is physically associated with the surface of the particle. If the antigen is a viral coat protein, then it may self-assemble to form the particle surface, such as an icosahedral virus-like particle. Typically, the antigen is in some way substantially anchored to the surface, for example through a covalent bond to a component of the surface. Alternatively or in addition, the antigen includes an anchoring domain which domain can be incorporated, covalently or noncovalently, into the general structure of the surface per se. In preferred embodiments, the physical association between the antigen and the surface of the particle is not mediated by an antigen-specific antibody or antigen-specific fragment thereof.

The antigen is capable of being bound by an antibody that binds specifically to the antigen. Importantly, the antigen, as present on the particle per se, is not bound to or by an antibody. Rather, as described herein, the antigen as present on the particle becomes bound by an antibody that binds specifically to the antigen upon administration of the particle to a subject, whereupon the subject's immune response to the antigen comprises binding of the antibody to the antigen, wherein such antibody binds specifically to the antigen.

By the phrase “binds specifically,” as used herein, is meant that a compound, e.g., a protein, a nucleic acid, an antibody, and the like, recognizes and binds a particular molecule, but does not substantially recognize or bind other molecules in a sample. For instance, the phrase “binds specifically” may characterize an antibody or a peptide inhibitor which recognizes and binds a cognate ligand (e.g., an anti-PD-1 antibody that binds with its cognate antigen, PD-1) in a sample, but does not substantially recognize or bind other molecules in the sample. Thus, under designated assay conditions, the specified binding moiety (e.g., an antibody or an antigen-binding portion thereof) binds preferentially to a particular target molecule and does not bind in a significant amount to other components present in a test sample. A variety of assay formats may be used to select an antibody that specifically binds a molecule of interest. For example, solid-phase ELISA immunoassay, immunoprecipitation, BIAcore and Western blot analysis are used to identify an antibody that specifically reacts with PD-1. Typically a specific or selective reaction will be at least twice background signal or noise, and more typically more than 10 times background.

Even more specifically, an antibody is said to “bind specifically” to an antigen when the equilibrium dissociation constant between the antibody and its antigen (K_{D}) is ≤1 μM, more preferably ≤100 nM, even more preferably ≤10 nM, and most preferably ≤1 nM.

In certain embodiments, the antibody binds the antigen with a K_{D} of less than or equal to 10⁻⁷ M. In certain embodiments, the antibody binds the antigen with a K_{D} of less than or equal to 10⁻⁸ M. In certain embodiments, the antibody binds the antigen with a K_{D} of less than or equal to 10⁻⁹M. In certain embodiments, the antibody binds the antigen with a K_{D} of less than or equal to 10⁻¹° M. In certain embodiments, the antibody binds the antigen with a K_{D} of less than or equal to 10⁻¹¹M. In certain embodiments, the antibody binds the antigen with a K_{D} of less than or equal to 10⁻¹²M. Methods for measuring K_{D} are well known in the art and include, for example, surface plasmon resonance (BIAcore, GE Healthcare Life Sciences).

As noted above, the antigen is capable of being bound by an antibody that binds specifically to the antigen. In preferred embodiments, the antibody comprises an Fc domain that is capable of being bound by FcR on immune cells. In preferred embodiments, the antibody comprises an Fcγ domain that is capable of being bound by FcγR on immune cells. In preferred embodiments, the antibody comprises an Fcγ domain that is capable of being bound and internalized by FcγRIIA expressed on pDC.

In certain embodiments, the interior of the particle comprises a payload or payload compound. In certain embodiments, the payload is substantially unassociated with, i.e., not covalently linked to, the inner aspect or inner face of the surface. For example, in certain embodiments at least about 90 percent of the payload is substantially unassociated with the inner aspect or inner face of the surface. In certain embodiments, the payload is partially associated with the inner aspect or inner face of the surface. For example, in certain embodiments about 20 percent of the payload is associated, covalently or non-covalently, with the inner aspect or inner face of the surface, while the remainder of the payload is unassociated with the inner aspect or inner face of the surface. In certain embodiments, the payload is substantially associated with the inner aspect or inner face of the surface. For example, in certain embodiments at least about 90 percent of the payload is associated with the inner aspect or inner face of the surface.

In certain embodiments, the payload compound is not an expressable nucleic acid molecule, e.g., a gene.

In certain embodiments, the payload compound is selected from the group consisting of therapeutic agents, immune modulating agents, immune activators, immune suppressors, imaging agents, and any combination thereof. In certain embodiments, the payload compound is a therapeutic agent. In certain embodiments, the payload compound is an immune modulating agent. In certain embodiments, the payload compound is an immune activator. In certain embodiments, the payload compound is an immune suppressor. In certain embodiments, the payload compound is an imaging agent.

In certain embodiments, the therapeutic agent is a synthetic nucleic acid. In certain embodiments the therapeutic agent is selected from the group consisting of antisense, RNAi, aptamers, antagomirs, microRNAs, and any combination thereof. In certain embodiments, the therapeutic agent is antisense. In certain embodiments, the therapeutic agent is RNAi. In certain embodiments, the therapeutic agent is an aptamer. In certain embodiments, the therapeutic agent is an antagomir, which for the purposes of this application includes any microRNA inhibitor or antagonist known in the art, including blockmirs (such as described in U.S. Pat. No. 8,691,965 to Møller). In certain embodiments, the therapeutic agent is a microRNA.

In certain embodiments, the payload compound is an immune activator.

In certain embodiments, the immune activator is a TLR agonist.

In certain embodiments, the TLR agonist is a TLR7 agonist. TLR7 recognizes single-stranded RNA in endosomes, which is a common feature of viral genomes which are internalized by macrophages and dendritic cells. In addition to single-stranded RNA, TLR7 also recognizes the imidazoquinoline imiquimod (3-(2-Methylpropyl)-3,5,8-triazatricyclo[7.4.0.0^{2,6}]trideca-1(9),2(6),4,7,10,12-hexaen-7-amine; Aldara® (3M)) and many other related and unrelated compounds known in the art.

In certain embodiments, the TLR agonist is a TLR9 agonist.

In certain embodiments, the TLR agonist is a synthetic CpG DNA oligonucleotide.

In certain embodiments, the immune activator induces type I IFN. In certain embodiments, the immune activator induces large amounts of type I IFN.

For example, certain preferred CpG ODN, such as CpG-A, CpG-C, and other CpG ODN well known in the art induce high or large amounts of type I IFN. Assays for measuring type I IFN are well known in the art and include in vitro enzyme-linked immunosorbent assay (ELISA) and cell-based assays. Without meaning to be limiting, large or high amounts of type I IFN can refer to greater than or equal to about 1000 pg/mL IFN-α as measured according to such in vitro assays. In certain embodiments, large or high amounts of type I IFN can refer to greater than or equal to about 2000 pg/mL IFN-α as measured according to such in vitro assays. In certain embodiments, large or high amounts of type I IFN can refer to greater than or equal to about 3000 pg/mL IFN-α as measured according to such in vitro assays. In certain embodiments, large or high amounts of type I IFN can refer to greater than or equal to about 4000 pg/mL IFN-α as measured according to such in vitro assays. In certain embodiments, large or high amounts of type I IFN can refer to greater than or equal to about 5,000 pg/mL IFN-α as measured according to such in vitro assays.

Preferred particles of the invention do not induce, or only weakly induce, complement activation, i.e., will have no or only very slight net negative (or positive) charge, and have a uniform curvature.

It is preferred that a priming dose of the particles of the invention should induce a very strong Ab response to the antigen (very high IgG Ab titer) so that when the therapeutic dose is administered, the immune complexes that form in the circulation (if the therapeutic dose is to be given IV) will be in high antibody excess so that the resulting immune complexes are very small and do not deposit significantly in peripheral tissues or induce high levels of complement activation. Thus it is preferred to avoid toxicity that could be dose-limiting, or may prevent the administration of doses high enough to induce systemic pDC activation where that is a desired therapeutic effect. To avoid allergic or anaphylactic responses to the particle, it is also preferred that the priming dose does not induce an IgE response, but rather induces a Th1-biased immune response, such as is induced by TLR7 and TLR9 agonists.

The therapeutic payload delivered in the particle of the invention can be chosen such that it will be active in only a subset of FcR-expressing cells. For example, a TLR9 or TLR7 agonist payload will only activate human pDC or B cells, and a CpG-A agonist for TLR9 provides a relative selectivity for pDC activation.

Preferred particles of the invention are under 200 nm in mean size, more preferably under 100 nm in mean size, and most preferably under 50 or approximately 30 nm, (and can readily be produced under GMP with a narrow size distribution).

Preferred particles of the invention are stable for prolonged storage in lyophilized form, or frozen at −80° C., more preferably at −20° C., even more preferably at 4° C., and most preferably at room temperature.

### CpG DNA

CpG oligonucleotides (CpG DNA; CpG ODN) contain specific sequences found to elicit an immune response. These specific sequences are referred to as “immunostimulatory motifs”, and the oligonucleotides that contain immunostimulatory motifs are referred to as “immunostimulatory oligonucleotide molecules” and equivalently, “immunostimulatory oligonucleotides”. Immunostimulatory oligonucleotides include at least one immunostimulatory motif, and preferably that motif is an internal motif. The term “internal immunostimulatory motif” refers to the position of the motif sequence within an oligonucleotide sequence which is at least one nucleotide longer (at both the 5′ and 3′ ends) than the motif sequence.

CpG oligonucleotides include at least one unmethylated CpG dinucleotide. An oligonucleotide containing at least one unmethylated CpG dinucleotide is an oligonucleotide molecule which contains a cytosine-guanine dinucleotide sequence (i.e., “CpG DNA” or DNA containing a 5′ cytosine linked by a phosphate bond to a 3′ guanine) and activates the immune system. The entire CpG oligonucleotide can be unmethylated or portions may be unmethylated, but at least the C of the 5′ CG 3′ must be unmethylated.

CpG ODN are generally about 8-100 nucleotides long. In certain embodiments, CpG ODN are about 8-50 nucleotides long, about 8-40 nucleotides long, about 8-30 nucleotides long, about 8-24 nucleotides long, about 8-20 nucleotides long, or about 8-16 nucleotides long.

By 2004, structure-activity relationship studies of CpG ODN had defined three families with distinct structural and biological characteristics (Hartmann, G. et al. (2003) Eur J Immunol 33:1633-1641; Marshall et al. (2003) J Leukocyte Biol 73:781-792; Vollmer et al. (2004) Eur J Immunol 34:251-262). Typical B-class CpG ODN (CpG-B) have a completely phosphorothioate backbone, do not form higher-ordered structures, and are strong B cell stimulators, inducing relatively high levels of IL-10 secretion, but induce relatively little NK activity or IFN-α secretion (Krieg, 2002, and Krieg, unpublished observations). B-class CpG ODN induce immune-suppressive counter-regulatory effects including not only the secretion of IL-10, but also the expression of IDO, which can promote the development of Treg cells in vitro (Moseman et al. (2004) J Immunol 173(7):4433-4442; Chen et al. (2008) J Immunol 181(8):5396-5404). The relevance of these in vitro data to in vivo tumor immunotherapy has been uncertain, and has not delayed the clinical development of B-class CpG ODN, but the present invention is based in part on a new discovery that these effects of B-class CpG ODN will suppress anti-tumor immune responses, which can be avoided using other classes of CpG ODN that are structurally designed not to activate the NF-κB pathway leading to IL-10 secretion.

The phosphorothioate backbone used in B-class CpG ODN has multiple complex effects on the resulting immune response compared to that seen with a CpG ODN with the same sequence but without a phosphorothioate backbone. One very important effect of the phosphorothioate (PS) backbone is protection against nuclease degradation. Completely PS-modified ODN are nearly completely stable in serum and tissues for at least 24 hr, whereas unmodified and unprotected ODN are degraded within a few minutes. In serum the major nuclease activity is a 3′ exonuclease against which CpG ODN can be protected with just 1 or a few PS linkages at the 3′ end of the ODN. But in tissues there also are 5′ exonucleases as well as endonucleases, and these can degrade native DNA that is not otherwise protected. Native DNA can be protected against exonucleases by circularization using techniques well described in the literature. See, for example, U.S. Pat. Nos. 8,017,591; 7,635,468; 7,074,772; 6,849,725; 6,451,593; and 6,451,563; and U.S. Published Patent Application No. 2003/0125279; the entire contents of all of which are hereby incorporated by reference. Alternatively or in addition, the native (i.e., otherwise unmodified and unprotected) ODN can be formulated in nanoparticles or other formulations well known in the art to block nuclease access to the ODN.

In general, native CpG DNA (phosphodiester) activates TLR9 in both B cells and pDC. B cells produce cytokine and start to proliferate (this is predominantly driven through NF-κB activation), but unless the TLR9 stimulation is sustained, the proliferation is usually modest, and relatively little stimulation of Ig secretion and class switching occurs. pDC are activated by native CpG DNA to secrete type I IFN and to express costimulatory receptors, but the magnitude of the stimulation depends critically on the form of the DNA. In contrast to these effects of native CpG DNA, B-class phosphorothioate CpG DNA provides a far more powerful and sustained TLR9 signal for B cells, inducing them to proliferate strongly and leading to Ig secretion and class switching as reported in the literature. But the phosphorothioate backbone has a very different effect on the TLR9-mediated pDC response, reducing substantially the type I IFN secretion (apparently through suppressing IRF7-mediated signaling), but usually still providing strong induction of costimulatory molecule expression. Thus, for the present invention, the use of native DNA usually will provide higher type I IFN responses and will be therapeutically effective as long as the native DNA is protected from degradation. From 1 to 3 phosphorothioate modifications can be added onto the 5′ and 3′ termini of native DNA to protect it from nuclease degradation without diminishing the type I IFN response.

The B-class of CpG oligonucleotides is represented by the formula:

5′ X₁CGX₂ 3′

wherein X₁ and X₂ are nucleotides. In some embodiments, X₁ may be adenine, guanine, or thymine and/or X₂ may be cytosine, adenine, or thymine.

The B-class of CpG oligonucleotides is also represented by the formula:

5′ X₁X₂CGX₃X₄ 3′

wherein X₁, X₂, X₃, and X₄ are nucleotides. X₂ may be adenine, guanine, or thymine. X₃ may be cytosine, adenine, or thymine.

The B-class of CpG oligonucleotides also includes oligonucleotides represented by at least the formula:

5′ N₁X₁X₂CGX₃X₄N₂ 3′

wherein X₁, X₂, X₃, and X₄ are nucleotides and N is any nucleotide and N₁ and N₂ are oligonucleotide sequences composed of from about 0-25 N's each. X₁X₂ may be a dinucleotide selected from the group consisting of: GpT, GpG, GpA, ApA, ApT, ApG, CpT, CpA, CpG, TpA, TpT, and TpG; and X₃X₄ may be a dinucleotide selected from the group consisting of: TpT, ApT, TpG, ApG, CpG, TpC, ApC, CpC, TpA, ApA, and CpA.

The B-class of CpG oligonucleotides is disclosed in PCT Published Patent Applications PCT/US95/01570 and PCT/US97/19791, and U.S. Pat. Nos. 6,194,388 and 6,239,116.

In contrast to the B-class CpG ODN, A-class CpG ODN (CpG-A) are potent activators of IFN-α secretion from plasmacytoid dendritic cells (pDC), and secondary activators of natural killer cells, but only weakly stimulate B cells, and induce very little IL-10 secretion. Canonical A-class CpG ODN contain polyG motifs at the 5′ and/or 3′ ends which are capable of forming complex higher-ordered structures known as G-tetrads and a central phosphodiester region containing one or more CpG motifs within a self-complementary palindrome (reviewed in (Krieg, 2006). For example, U.S. Pat. Nos. 6,949,520 and 7,776,344 show that in certain preferred embodiments the A-class CpG ODN has a sequence corresponding to any of the following:

(SEQ ID NO: 1)
ggGGTCAACGTTGAgggggG;
(SEQ ID NO: 2)
ggGGGACGATCGTCgggggG;
(SEQ ID NO: 3)
ggGGGACGATATCGTCgggggG;
(SEQ ID NO: 4)
ggGGGACGACGTCGTCgggggG;
(SEQ ID NO: 5)
ggGGGACGAGCTGCTCgggggG;
(SEQ ID NO: 6)
ggGGGACGTACGTCgggggG;
(SEQ ID NO: 7)
ggGGGACGATCGTTGgggggG;
(SEQ ID NO: 8)
ggGGAACGATCGTCggggG;
(SEQ ID NO: 9)
ggGGGGACGATCGTCgggggG;
(SEQ ID NO: 10)
ggGGGACGATCGTCGgggggG;
(SEQ ID NO: 11)
ggGGGTCATCGATGAgggggG;
(SEQ ID NO: 12)
ggGGTCGTCGACGAgggggG;
(SEQ ID NO: 13)
ggGGTCGTTCGAACGAgggggG;
(SEQ ID NO: 14)
ggGGACGTTCGAACGTgggggG;
(SEQ ID NO: 15)
ggGGAACGACGTCGTTgggggG;
(SEQ ID NO: 16)
ggGGAACGTACGTCgggggG;
(SEQ ID NO: 17)
ggGGAACGTACGTACGTTgggggG;
(SEQ ID NO: 18)
ggGGTCACCGGTGAgggggG;
(SEQ ID NO: 19)
ggGGTCGACGTACGTCGAgggggG;
(SEQ ID NO: 20)
ggGGACCGGTACCGGTgggggG;
(SEQ ID NO: 21)
ggGTCGACGTCGAgggggG;
(SEQ ID NO: 22)
ggGGTCGACGTCGagggg;
(SEQ ID NO: 23)
ggGGAACGTTAACGTTgggggG;
(SEQ ID NO: 24)
ggGGACGTCGACGTggggG;
(SEQ ID NO: 25)
ggGGGTCGTTCGTTgggggG;
(SEQ ID NO: 26)
ggGACGATCGTCGgggggG;
(SEQ ID NO: 27)
ggGTCGTCGACGAggggggG;
(SEQ ID NO: 28)
ggTCGTCGACGAGgggggG;
(SEQ ID NO: 29)
ggGGACGATCGTCGgggggG;
(SEQ ID NO: 30)
ggGGTCGACGTCGACGTCGAGgggggG;
and
(SEQ ID NO: 31)
ggGGACGACGTCGTGgggggG,

wherein each lower case letter represents a nucleotide linked to its 3′-adjacent nucleotide by a phosphorothioate (PS) linkage; and each upper case letter represents a nucleotide linked to its 3′-adjacent nucleotide (if present) by a phosphodiester (PO) linkage, except that the 3′-terminal nucleotide is represented by an upper case letter since it has no 3′-adjacent nucleotide.

In certain embodiments, an A-class CpG ODN for use in accordance with the methods of the instant invention has a sequence provided as: 5′-GGGGGGGGGGGACGATCGTCGGGGGGGGGG-3′ (SEQ ID NO:32); also referred to herein as “G10”). Such oligonucleotide and formulations thereof useful in accordance with the present invention are described in WO 2003/024481; US 2003/0099668; US 2012/0301499; WO 2004/084940; U.S. Pat. No. 7,517,520; US 2010/0098722; WO 2007/068747; US 2007/0184068; U.S. Pat. No. 8,574,564; WO 2007/144150; U.S. Pat. No. 8,541,559; WO 2008/073960; and U.S. Pat. No. 8,586,728, the entire contents of each of which is incorporated herein by reference.

The structure of C-class CpG ODN is typically based on a phosphorothioate backbone, but is distinct in that the CpG motifs are followed by a 3′ palindrome, which may form a duplex. C-class CpG ODN (CpG-C) are described in U.S. Pat. No. 7,566,703 to Krieg et al.; U.S. Pat. No. 8,198,251 to Vollmer et al.; and U.S. Pat. No. 8,834,900 to Krieg et al. The C-class CpG ODN have immune properties intermediate between the A and B classes (Hartmann, G. et al. 2003; Marshall et al., 2003; Marshall et at, 2005; Vollmer et at, 2004).

Examples of C-class ODN include:

(SEQ ID NO: 33)
TCGTCGTTTTCGGCGCGCGCCG;
(SEQ ID NO: 34)
TCGTCGTTTTCGGCGGCCGCCG;
(SEQ ID NO: 35)
TCGTCGTTTTCGGCGCGCCGCG;
(SEQ ID NO: 36)
TCGTCGTTTTCGGCGCCGGCCG;
(SEQ ID NO: 37)
TCGTCGTTTTCGGCCCGCGCGG;
(SEQ ID NO: 38)
TCGTCGTTTTCGGCGCGCGCCGTTTTT;
(SEQ ID NO: 39)
TCCTGACGTTCGGCGCGCGCCG;
(SEQ ID NO: 40)
TZGTZGTTTTZGGZGZGZGZZG;
(SEQ ID NO: 41)
TCCTGACGTTCGGCGCGCGCCC;
(SEQ ID NO: 42)
TCGGCGCGCGCCGTCGTCGTTT;
(SEQ ID NO: 43)
TCGTCGTTTTCGGCGGCCGACG;
(SEQ ID NO: 44)
TCGTCGTTTTCGTCGGCCGCCG;
(SEQ ID NO: 45)
TCGTCGTTTTCGACGGCCGCCG;
(SEQ ID NO: 46)
TCGTCGTTTTCGGCGGCCGTCG;
(SEQ ID NO: 47)
TCGTCGTTTCGACGGCCGTCG;
(SEQ ID NO: 48)
TCGTCGTTTCGACGATCGTCG;
(SEQ ID NO: 49)
TCGTCGTTTCGACGTACGTCG;
(SEQ ID NO: 50)
TCGTCGCGACGGCCGTCG;
(SEQ ID NO: 51)
TCGTCGCGACGATCGTCG;
(SEQ ID NO: 52)
TCGTCGCGACGTACGTCG;
(SEQ ID NO: 53)
TCGTTTTTTTCGACGGCCGTCG;
(SEQ ID NO: 54)
TCGTTTTTTTCGACGATCGTCG;
and
(SEQ ID NO: 55)
TCGTTTTTTTCGACGTACGTCG,

wherein each Z is 5-methylcytosine.

The CpG oligonucleotides may be partially resistant to degradation (e.g., are stabilized). A “stabilized oligonucleotide molecule” shall mean an oligonucleotide that is relatively resistant to in vivo degradation (e.g. via an exo- or endo-nuclease). Oligonucleotide stabilization can be accomplished via backbone modifications. Oligonucleotides having phosphorothioate linkages provide maximal protection for the oligonucleotide from degradation by intracellular exo- and endo-nucleases. Other modified oligonucleotides include phosphodiester modified oligonucleotides, combinations of phosphodiester and phosphorothioate oligonucleotide, methylphosphonate, methylphosphorothioate, phosphorodithioate, p-ethoxy, and combinations thereof. Oligonucleotides which contain diol, such as tetraethyleneglycol or hexaethyleneglycol, at either or both termini have also been shown to be substantially resistant to nuclease degradation. Circular ODN are protected against exonuclease degradation. For example, the Mologen double stem-loop immunomodulator MGN1703 (formerly dSLIM-30L1) is a covalently closed 116-nucleotide dumbbell-shaped CpG-containing phosphodiester backbone oligonucleotide having the sequence 5′-AGGTGGTAACCCCTAGGGGTTACCACCTTCATTGGAAAACGTTCTTCGGGGC GTTCTTAGGTGGTAACCCCTAGGGGTTACCACCTTCATTGGAAAACGTTCTTCG GGGCGTTCTT-3′ (SEQ ID NO:56). Schmidt, M. et al. (2006) Allergy 61:56-63; Kapp, K. et al. (2014) Mol Ther Nucleic Acids 3:e170.

### TLR Antagonists

In contrast to CpG DNA, certain other oligonucleotides are TLR antagonists, including in particular antagonists of TLR7, TLR8, and/or TLR9. Such antagonists are referred to as S-class oligonucleotides or S-class ODN, and they are described, for example, in U.S. Pat. No. 9,260,719 to Kandimalla et al., U.S. Pat. No. 9,206,430 to Kandimalla et al., U.S. Pat. No. 8,987,221 to Zhu et al., U.S. Pat. No. 8,962,579 to Barrat et al., U.S. Pat. No. 8,940,310 to Barrat et al., and U.S. Pat. No. 8,759,305 to Barrat et al.; and U.S. Patent Application Publication No. 2015/0344884 to Uhlmann et at, the entire contents of each of which is incorporated herein by reference.

### Checkpoint Inhibitors

A. Anti-PD-1

Programmed death-1 receptor (PD-1), also known as CD279, is a type 1 membrane protein expressed on activated T cells (including CD8⁺ T cells), B cells, and macrophages. Its cognate ligands are PD-L1 and PD-L2, and binding of PD-1 particularly by PD-L1 blocks “Signal 3” in T cells and potently inhibits the effector arm of an adaptive immune response, for example by leading to the death of T cells expressing PD-1.

In humans, PD-1 is a 268-amino acid polypeptide having an amino acid sequence published as GenBank Accession No. NP_005009. The protein includes an extracellular IgV domain, transmembrane domain, and intracellular domain having two phosphorylation sites.

The K_{D} for interaction between PD-1 and PD-L1 is 770 nM.

In preferred embodiments of the invention, the antibody inhibits binding between PD-1 and PD-L1. Preferably, the antibody can inhibit binding with PD-L1 with an IC₅₀ of about 100 nM or lower; more preferably, about 10 nM or lower, for example about 5 nM or lower; yet more preferably, about 2 nM or lower; or even more preferably, for example, about 1 nM or lower.

Further, in another embodiment, the anti-PD-1 antibody has a binding affinity for PD-1 that is at least as strong as that of PD-L1. In certain embodiments, the anti-PD-1 antibody has a binding affinity for PD-1 that is at least 10 times as strong as that of PD-L1. In certain embodiments, the anti-PD-1 antibody has a binding affinity for PD-1 that is at least 100 times as strong as that of PD-L1. In certain embodiments, the anti-PD-1 antibody has a binding affinity for PD-1 that is at least 1000 times as strong as that of PD-L1.

Anti-PD-1 antibodies are known in the art and include, for example, those disclosed in U.S. Pat. No. 6,808,710 to Wood et al., U.S. Pat. No. 7,488,802 to Collins et al., and U.S. Pat. No. 8,728,474 to Honjo et al. Anti-PD-1 antibodies are commercially available as pembrolizumab (formerly known as lambrolizumab and MK-3475, KEYTRUDA®, Merck, K_{D} 29 pM) and nivolumab (OPDIVO®, Bristol-Myers Squibb, K_{D} 2.6 nM). Additional anti-PD-1 antibodies currently under development include pidilizumab (CT-011, Cure Tech).

B. Anti-PD-L1

Programmed death-ligand 1 receptor (PD-L1), also known as CD274 and B7 homolog 1 (B7-H1), is a type 1 membrane protein expressed on activated T cells (including CD8⁺ T cells and so-called tumor-infiltrating lymphocytes (TIL cells)), B cells, macrophages, and dendritic cells, as well as on many types of tumor cells. Its cognate ligands are PD-1 and B7.1 (CD80), and binding of PD-1 by PD-L1 blocks “Signal 3” in T cells and can potently inhibit the T cell effector functions mediating an adaptive immune response, for example by leading to the death of T cells expressing PD-1.

PD-L1 expression is upregulated on T cells, NK cells, macrophages, myeloid dendritic cells, B cells, epithelial cells, and vascular endothelial cells in response to interferon gamma (IFN-γ). PD-L1 expression is also upregulated on tumors, e.g., renal cell carcinoma and ovarian cancer, in response to IFN-γ.

In humans, PD-L1 is expressed in either of two isoforms, a longer isoform a or a shorter isoform b. Isoform a is a 290-amino acid polypeptide having an amino acid sequence published as GenBank Accession No. NP_054862; the mature peptide comprises amino acid residues 19-290, with residues 239-259 representing the transmembrane domain Isoform b is a 176-amino acid polypeptide having an amino acid sequence published as GenBank NP_001254635; the mature peptide comprises amino acid residues 19-259.

As mentioned above, the K_{D} for interaction between PD-1 and PD-L1 is 770 nM.

In preferred embodiments of the invention, the antibody inhibits binding between PD-1 and PD-L1. Preferably, the antibody can inhibit binding with PD-1 with an IC₅₀ of about 100 nM or lower; more preferably, about 10 nM or lower, for example about 5 nM or lower; yet more preferably, about 2 nM or lower; or even more preferably, for example, about 1 nM or lower.

Further, in another embodiment, the anti-PD-L1 antibody has a binding affinity for PD-L1 that is at least as strong as that of PD-1. In certain embodiments, the anti-PD-L1 antibody has a binding affinity for PD-L1 that is at least 10 times as strong as that of PD-1. In certain embodiments, the anti-PD-L1 antibody has a binding affinity for PD-L1 that is at least 100 times as strong as that of PD-1. In certain embodiments, the anti-PD-L1 antibody has a binding affinity for PD-L1 that is at least 1000 times as strong as that of PD-1.

Anti-PD-L1 antibodies are known in the art and include, for example, those disclosed in U.S. Pat. No. 7,943,743 to Korman et al. While no anti-PD-L1 antibodies are yet approved by the FDA for commercialization in the United States, several anti-PD-L1 antibodies are currently under development in human clinical trials, including MPDL3280A (Genetech/Roche, K_{D} 0.4 nM), BMS-936559 (Bristol-Myers Squibb), and MEDI-4736 (AstraZeneca).

C. Anti-CTLA-4

Cytotoxic T-lymphocyte-associated protein 4 (CTLA-4), also known as CTLA4 or CD152, is a membrane protein expressed on T cells and regulatory T cells (Treg). Its cognate ligands include B7-1 (CD80) and B7-2 (CD86) on antigen-presenting cells (APC). Binding of B7-1 or B7-2 by CTLA-4 blocks “Signal 2” in T cells and inhibits the initiation of an adaptive immune response.

In humans, CTLA-4 is encoded in various isoforms, including one with an amino acid sequence published as GenBank Accession No. NP_001032720.

A preferred anti-CTLA-4 antibody is an antibody that specifically binds to human CTLA-4. More particularly, the anti-CTLA-4 antibody specifically binds to an epitope in the extracellular domain of human CTLA-4 and inhibits binding between CTLA-4 and one or both of its cognate ligands B7-1 and B7-2.

A preferred anti-CTLA-4 antibody is a human antibody that specifically binds to human CTLA-4. More particularly, the anti-CTLA-4 antibody specifically binds to an epitope in the extracellular domain of human CTLA-4 and inhibits binding between CTLA-4 and one or both of its cognate ligands B7-1 and B7-2. Exemplary human anti-CTLA-4 antibodies are described in detail in International Application No. PCT/US99/30895, published on Jun. 29, 2000 as WO 00/37504; European Patent Appl. No. EP 1262193 A1, published Apr. 12, 2002; U.S. patent application Ser. No. 09/472,087, now issued as U.S. Pat. No. 6,682,736, to Hanson et al.; U.S. patent application Ser. No. 09/948,939, published as US 2002/0086014; U.S. patent application Ser. No. 11/988,396, published as US 2009/0117132; and U.S. patent application Ser. No. 13/168,206, published as US 2012/0003179, the entire disclosures of which are incorporated herein by reference. Such antibodies include, but are not limited to, 3.1.1, 4.1.1, 4.8.1, 4.10.2, 4.13.1, 4.14.3, 6.1.1, 11.2.1, 11.6.1, 11.7.1, 12.3.1.1, and 12.9.1.1, as well as MDX-010. Human antibodies provide a substantial advantage in the treatment methods of the present invention, as they are expected to minimize the immunogenic and allergic responses that are associated with use of non-human antibodies in human patients.

Anti-CTLA-4 antibodies specifically include ipilimumab (YERVOY®, Bristol-Myers Squibb).

D. Other Checkpoint Inhibitors

In addition to those listed above, other checkpoints are known in the art and their inhibitors are included in the invention. For example, BTLA provides a negative signal in response to HVEM, and TIM3 provides a negative signal in response to Gal9. Adenosine can trigger suppressive effects through the adenosine A2a receptor, and IDO and TDO are well known immunosuppressive pathways thought to be involved in anti-tumor immunity. LAG3 binds to MHC class II with higher affinity than CD4. LAG3 negatively regulates cellular proliferation, activation, and homeostasis of T cells, in a fashion similar to CTLA-4 and PD-1, and it has been reported to play a role in Treg suppressive function. LAG3 also helps maintain CD8⁺ T cells in a tolerogenic state and, working with PD-1, helps maintain CD8 exhaustion during chronic viral infection. LAG3 is known to be involved in the maturation and activation of dendritic cells. Additional checkpoint inhibitors for use in the invention include, without limitation, antibodies and antigen-binding fragments thereof, capable of binding specifically to any one or more of BTLA, TIM3, and LAG3. Also contemplated by the invention are bispecific antibodies and bispecific antigen-binding fragments thereof which are capable of binding specifically to any one or more of BTLA, TIM3, and LAG3.

### Methods of the Invention

An aspect of the invention is a method of modulating an immune response. The method includes the step of systemically administering to a subject in need thereof an effective amount of a particle comprising a surface and an interior, wherein the surface of the particle comprises an antigen, the interior of the particle comprises an immune modulating agent, and the subject previously was primed to mount an antibody response to the antigen, to modulate an immune response of the subject.

A “subject” as used herein refers to a living mammal. In certain embodiments, a subject is a mouse, rat, guinea pig, rabbit, sheep, goat, cat, dog, horse, cow, or non-human primate. In certain embodiments, a subject is a human.

An “effective amount” as used herein is an amount that is sufficient to achieve a desired biological effect. A “therapeutically effective amount” as used herein is an amount that is sufficient to achieve a desired therapeutic effect.

A “subject primed to mount an antibody response to the antigen” as used herein refers to a subject whose immune system is not naïve to the antigen. In certain embodiments, the subject has circulating antibodies specific for the antigen. In certain embodiments, the subject has memory B cells or plasma cells specific for the antigen. The subject may have been previously exposed to the antigen either naturally, by having an infection with an infectious agent (e.g., a virus) comprising the antigen, or through previous immunization against such an infectious disease.

In certain embodiments, the particle is a non-naturally occurring particle.

In certain embodiments, the particle is selected from the group consisting of liposomes, virus-like particles, and lipid nanoparticles. The particle generally is roughly spherical in shape but it may take other shapes including, for example, polyhedrons, oblates, cylinders, boxes, cubes, cuboids, pyramids, and irregular three-dimensional shapes.

The particle generally is about 10 nm to about 2000 nm in its greatest diameter. In certain embodiments, the particle is about 10 nm to about 100 nm in its greatest diameter. In certain embodiments, the particle is about 10 nm to about 200 nm in its greatest diameter. In certain embodiments, the particle is about 10 nm to about 300 nm in its greatest diameter. In certain embodiments, the particle is about 10 nm to about 400 nm in its greatest diameter. In certain embodiments, the particle is about 10 nm to about 500 nm in its greatest diameter. In certain embodiments, the particle is about 10 nm to about 1000 nm in its greatest diameter. In certain embodiments, the particle is about 100 nm to about 200 nm in its greatest diameter. In certain embodiments, the particle is about 100 nm to about 300 nm in its greatest diameter. In certain embodiments, the particle is about 100 nm to about 400 nm in its greatest diameter. In certain embodiments, the particle is about 100 nm to about 500 nm in its greatest diameter. In certain embodiments, the particle is about 100 nm to about 1000 nm in its greatest diameter. In certain embodiments, the particle is about 100 nm to about 2000 nm in its greatest diameter. In certain embodiments, the particle is about 200 nm to about 500 nm in its greatest diameter. In certain embodiments, the particle is about 200 nm to about 1000 nm in its greatest diameter. In certain embodiments, the particle is about 200 nm to about 2000 nm in its greatest diameter. In certain embodiments, the particle is about 500 nm to about 1000 nm in its greatest diameter. In certain embodiments, the particle is about 500 nm to about 2000 nm in its greatest diameter. In certain embodiments, the particle is about 1000 nm to about 2000 nm in its greatest diameter.

The surface of the particle can be comprised of any biocompatible material, and, apart from the second antigen, it can be homogeneous or heterogeneous in its composition.

In certain embodiments, the antigen is selected from the group consisting of viral antigens, bacterial antigens, tumor antigens, and tumor neoantigens. In certain embodiments, the antigen is a viral antigen. In certain embodiments, the antigen is a bacterial antigen. In certain embodiments, the antigen is a tumor antigen. In certain embodiments, the antigen is a tumor neoantigen. Examples of each these types of antigen are well known in the art.

The antigen forms or is physically associated with the surface of the particle. Typically, the antigen is in some way substantially anchored to the surface, for example through a covalent bond to a component of the surface. Alternatively or in addition, the antigen includes an anchoring domain which domain can be incorporated, covalently or noncovalently, into the general structure of the surface per se. In preferred embodiments, the physical association between the antigen and the surface of the particle is not mediated by an antigen-specific antibody or antigen-specific fragment thereof.

The interior of the particle comprises an immune modulating agent. In certain embodiments, the immune modulating agent is substantially unassociated with the inner aspect or inner face of the surface. For example, in certain embodiments at least about 90 percent of the immune modulating agent is substantially unassociated with the inner aspect or inner face of the surface. In certain embodiments, the immune modulating agent is partially associated with the inner aspect or inner face of the surface. For example, in certain embodiments about 20 percent of the immune modulating agent is associated with the inner aspect or inner face of the surface, while the remainder of the immune modulating agent is unassociated with the inner aspect or inner face of the surface. In certain embodiments, the immune modulating agent is substantially associated with the inner aspect or inner face of the surface. For example, in certain embodiments at least about 90 percent of the immune modulating agent is associated with the inner aspect or inner face of the surface.

In certain embodiments, the immune modulating agent is not an expressable nucleic acid molecule, e.g., a gene.

In certain embodiments, the immune modulating agent is selected from the group consisting of therapeutic agents, immune modulating agents, immune activators, immune suppressors, imaging agents, and any combination thereof. In certain embodiments, the immune modulating agent is a therapeutic agent. In certain embodiments, the immune modulating agent is an immune activator. In certain embodiments, the immune modulating agent is an immune suppressor. In certain embodiments, the immune modulating agent is an imaging agent.

In certain embodiments, the therapeutic agent is selected from the group consisting of antisense, RNAi, aptamers, antagomirs, microRNAs, and any combination thereof. In certain embodiments, the therapeutic agent is antisense. In certain embodiments, the therapeutic agent is RNAi. In certain embodiments, the therapeutic agent is an aptamer. In certain embodiments, the therapeutic agent is an antagomir. In certain embodiments, the therapeutic agent is a microRNA.

In certain embodiments, the immune modulating agent is an immune activator.

In certain embodiments, the immune activator is a TLR agonist.

In certain embodiments, the TLR agonist is a TLR7 agonist.

In certain embodiments, the TLR agonist is a TLR9 agonist.

In certain embodiments, the TLR agonist is a synthetic CpG DNA oligonucleotide.

In certain embodiments, the immune modulating agent is an immune suppressor.

In certain embodiments, the immune suppressor is a TLR antagonist.

In certain embodiments, the TLR antagonist is an S-class ODN.

In certain embodiments, the TLR antagonist is a TLR7 antagonist.

In certain embodiments, the TLR antagonist is a TLR9 antagonist.

In certain embodiments, the particle is administered intravenously.

An aspect of the invention is a method of modulating an immune response in a subject. The method includes the steps of:

immunogenically administering to a subject in need thereof an effective amount of a first particle comprising a surface and an interior, wherein the surface of the first particle comprises an antigen, and the interior of the first particle optionally comprises a first immune modulating agent, to immunize the subject against the antigen; and

systemically administering to the subject an effective amount of a second particle comprising a surface and an interior, wherein the surface of the second particle comprises the antigen, and the interior of the second particle comprises a second immune modulating agent, to modulate an immune response of the subject.

In certain embodiments, the first particle is a non-naturally occurring particle.

In certain embodiments, the first particle is selected from the group consisting of liposomes, virus-like first particles, and lipid nanoparticles. The first particle generally is roughly spherical in shape but it may take other shapes including, for example, polyhedrons, oblates, cylinders, boxes, cubes, cuboids, pyramids, and irregular three-dimensional shapes.

The first particle generally is about 10 nm to about 2000 nm in its greatest diameter. In certain embodiments, the first particle is about 10 nm to about 100 nm in its greatest diameter. In certain embodiments, the first particle is about 10 nm to about 200 nm in its greatest diameter. In certain embodiments, the first particle is about 10 nm to about 300 nm in its greatest diameter. In certain embodiments, the first particle is about 10 nm to about 400 nm in its greatest diameter. In certain embodiments, the first particle is about 10 nm to about 500 nm in its greatest diameter. In certain embodiments, the first particle is about 10 nm to about 1000 nm in its greatest diameter. In certain embodiments, the first particle is about 100 nm to about 200 nm in its greatest diameter. In certain embodiments, the first particle is about 100 nm to about 300 nm in its greatest diameter. In certain embodiments, the first particle is about 100 nm to about 400 nm in its greatest diameter. In certain embodiments, the first particle is about 100 nm to about 500 nm in its greatest diameter. In certain embodiments, the first particle is about 100 nm to about 1000 nm in its greatest diameter. In certain embodiments, the first particle is about 100 nm to about 2000 nm in its greatest diameter. In certain embodiments, the first particle is about 200 nm to about 500 nm in its greatest diameter. In certain embodiments, the first particle is about 200 nm to about 1000 nm in its greatest diameter. In certain embodiments, the first particle is about 200 nm to about 2000 nm in its greatest diameter. In certain embodiments, the first particle is about 500 nm to about 1000 nm in its greatest diameter. In certain embodiments, the first particle is about 500 nm to about 2000 nm in its greatest diameter. In certain embodiments, the first particle is about 1000 nm to about 2000 nm in its greatest diameter.

The surface of the first particle can be comprised of any biocompatible material, and, apart from the antigen of the first particle, it can be homogeneous or heterogeneous in its composition.

In certain embodiments, the antigen is selected from the group consisting of viral antigens, bacterial antigens, tumor antigens, and tumor neoantigens. In certain embodiments, the antigen is a viral antigen. In certain embodiments, the antigen is a bacterial antigen. In certain embodiments, the antigen is a tumor antigen. In certain embodiments, the antigen is a tumor neoantigen. Examples of each these types of antigen are well known in the art.

The antigen forms or is physically associated with the surface of the first particle. Typically, the antigen is in some way substantially anchored to the surface of the first particle, for example through a covalent bond to a component of the surface of the first particle. Alternatively or in addition, the antigen includes an anchoring domain which domain can be incorporated, covalently or noncovalently, into the general structure of the surface per se of the first particle. In preferred embodiments, the physical association between the antigen and the surface of the first particle is not mediated by an antigen-specific antibody or antigen-specific fragment thereof.

In certain embodiments, the interior of the first particle comprises a first immune modulating agent.

In certain embodiments, the first immune modulating agent is substantially unassociated with the inner aspect or inner face of the surface of the first particle. For example, in certain embodiments at least about 90 percent of the first immune modulating agent is substantially unassociated with the inner aspect or inner face of the surface of the first particle. In certain embodiments, the first immune modulating agent is partially associated with the inner aspect or inner face of the surface of the first particle. For example, in certain embodiments about 20 percent of the first immune modulating agent is associated with the inner aspect or inner face of the surface of the first particle, while the remainder of the first immune modulating agent is unassociated with the inner aspect or inner face of the surface of the first particle. In certain embodiments, the first immune modulating agent is substantially associated with the inner aspect or inner face of the surface of the first particle. For example, in certain embodiments at least about 90 percent of the first immune modulating agent is associated with the inner aspect or inner face of the surface of the first particle.

In certain embodiments, the first immune modulating agent is not an expressable nucleic acid molecule, e.g., a gene.

In certain embodiments, the first immune modulating agent is selected from the group consisting of therapeutic agents, immune activators, and any combination thereof. In certain embodiments, the first immune modulating agent is a therapeutic agent. In certain embodiments, the first immune modulating agent is an immune activator.

In certain embodiments, the first immune modulating agent is a therapeutic agent, and said therapeutic agent is selected from the group consisting of antisense, RNAi, aptamers, antagomirs, microRNAs, and any combination thereof. In certain embodiments, said therapeutic agent is antisense. In certain embodiments, said therapeutic agent is RNAi. In certain embodiments, said therapeutic agent is an aptamer. In certain embodiments, said therapeutic agent is an antagomir. In certain embodiments, said therapeutic agent is a microRNA.

In certain embodiments, the first immune modulating agent is an immune activator. In certain embodiments, said immune activator is a TLR agonist. In certain embodiments, said TLR agonist is a TLR7 agonist. In certain embodiments, said TLR agonist is a TLR9 agonist. In certain embodiments, said TLR agonist is a synthetic CpG DNA oligonucleotide.

In certain embodiments, the second particle is a non-naturally occurring particle.

In certain embodiments, the second particle is selected from the group consisting of liposomes, virus-like second particles, and lipid nanoparticles. The second particle generally is roughly spherical in shape but it may take other shapes including, for example, polyhedrons, oblates, cylinders, boxes, cubes, cuboids, pyramids, and irregular three-dimensional shapes.

The second particle generally is about 10 nm to about 2000 nm in its greatest diameter. In certain embodiments, the second particle is about 10 nm to about 100 nm in its greatest diameter. In certain embodiments, the second particle is about 10 nm to about 200 nm in its greatest diameter. In certain embodiments, the second particle is about 10 nm to about 300 nm in its greatest diameter. In certain embodiments, the second particle is about 10 nm to about 400 nm in its greatest diameter. In certain embodiments, the second particle is about 10 nm to about 500 nm in its greatest diameter. In certain embodiments, the second particle is about 10 nm to about 1000 nm in its greatest diameter. In certain embodiments, the second particle is about 100 nm to about 200 nm in its greatest diameter. In certain embodiments, the second particle is about 100 nm to about 300 nm in its greatest diameter. In certain embodiments, the second particle is about 100 nm to about 400 nm in its greatest diameter. In certain embodiments, the second particle is about 100 nm to about 500 nm in its greatest diameter. In certain embodiments, the second particle is about 100 nm to about 1000 nm in its greatest diameter. In certain embodiments, the second particle is about 100 nm to about 2000 nm in its greatest diameter. In certain embodiments, the second particle is about 200 nm to about 500 nm in its greatest diameter. In certain embodiments, the second particle is about 200 nm to about 1000 nm in its greatest diameter. In certain embodiments, the second particle is about 200 nm to about 2000 nm in its greatest diameter. In certain embodiments, the second particle is about 500 nm to about 1000 nm in its greatest diameter. In certain embodiments, the second particle is about 500 nm to about 2000 nm in its greatest diameter. In certain embodiments, the second particle is about 1000 nm to about 2000 nm in its greatest diameter.

The surface of the second particle can be comprised of any biocompatible material, and, apart from the antigen, it can be homogeneous or heterogeneous in its composition.

The antigen forms or is physically associated with the surface of the second particle. Typically, the antigen is in some way substantially anchored to the surface of the second particle, for example through a covalent bond to a component of the surface of the second particle. Alternatively or in addition, the antigen includes an anchoring domain which domain can be incorporated, covalently or noncovalently, into the general structure of the surface per se of the second particle. In preferred embodiments, the physical association between the antigen and the surface of the second particle is not mediated by an antigen-specific antibody or antigen-specific fragment thereof.

The interior of the second particle comprises a second immune modulating agent.

In certain embodiments, the second immune modulating agent is substantially unassociated with the inner aspect or inner face of the surface of the second particle. For example, in certain embodiments at least about 90 percent of the second immune modulating agent is substantially unassociated with the inner aspect or inner face of the surface of the second particle. In certain embodiments, the second immune modulating agent is partially associated with the inner aspect or inner face of the surface of the second particle. For example, in certain embodiments about 20 percent of the second immune modulating agent is associated with the inner aspect or inner face of the surface of the second particle, while the remainder of the second immune modulating agent is unassociated with the inner aspect or inner face of the surface of the second particle. In certain embodiments, the second immune modulating agent is substantially associated with the inner aspect or inner face of the surface of the second particle. For example, in certain embodiments at least about 90 percent of the second immune modulating agent is associated with the inner aspect or inner face of the surface of the second particle.

In certain embodiments, the second immune modulating agent is not an expressable nucleic acid molecule, e.g., a gene.

In certain embodiments, the second immune modulating agent is selected from the group consisting of therapeutic agents, immune activators, immune suppressors, and any combination thereof. In certain embodiments, the second immune modulating agent is a therapeutic agent. In certain embodiments, the second immune modulating agent is an immune activator. In certain embodiments, the second immune modulating agent is an immune suppressor.

In certain embodiments, the second immune modulating agent is a therapeutic agent, and said therapeutic agent is selected from the group consisting of antisense, RNAi, aptamers, antagomirs, microRNAs, and any combination thereof. In certain embodiments, said therapeutic agent is antisense. In certain embodiments, said therapeutic agent is RNAi. In certain embodiments, said therapeutic agent is an aptamer. In certain embodiments, said therapeutic agent is an antagomir. In certain embodiments, said therapeutic agent is a microRNA.

In certain embodiments, the second immune modulating agent is an immune activator. In certain embodiments, said immune activator is a TLR agonist. In certain embodiments, said TLR agonist is a TLR7 agonist. In certain embodiments, said TLR agonist is a TLR9 agonist. In certain embodiments, said TLR agonist is a synthetic CpG DNA oligonucleotide.

In certain embodiments, the second immune modulating agent is an immune suppressor.

In certain embodiments, the second immune modulating agent is a TLR antagonist.

In certain embodiments, the second immune modulating agent is an S-class ODN.

In certain embodiments, the second immune modulating agent is a TLR7 antagonist.

In certain embodiments, the second immune modulating agent is a TLR9 antagonist.

In certain embodiments, the first particle and the second particle are the same.

In certain embodiments, the first particle and the second particle are different.

In certain embodiments, the first immune modulating agent and the second immune modulating agent are the same.

In certain embodiments, the first immune modulating agent and the second immune modulating agent are different.

In certain embodiments, the first particle is administered locally.

In certain embodiments, the first particle is administered subcutaneously or intramuscularly.

In certain embodiments, the second particle is administered intravenously.

In certain embodiments, the first particle is administered subcutaneously, and second particle is administered intravenously.

In certain embodiments, the first particle is administered intramuscularly, and second particle is administered intravenously.

An aspect of the invention is a method of treating a disease or condition. The method includes the step of:

systemically administering to a subject having a disease or condition an effective amount of a particle comprising a surface and an interior, wherein the surface of the particle comprises an antigen, the interior of the particle comprises an immune modulating agent, and the subject is primed to mount an antibody response to the antigen, to modulate an immune response of the subject, thereby treating the disease or condition.

As used herein, to “treat” means reducing the frequency with which symptoms of a disease (i.e., tumor growth and/or metastasis, or other effect mediated by the numbers and/or activity of immune cells, and the like) are experienced by a patient. Treatment may be prophylactic (to prevent or delay the onset of the disease, or to prevent the manifestation of clinical or subclinical symptoms thereof) or therapeutic suppression or alleviation of symptoms after the manifestation of the disease. The term “treat” includes the administration of the compounds or agents of the present invention to (i) prevent or delay the onset of the symptoms, complications, or biochemical indicia of, (ii) alleviate the symptoms of, and/or (iii) inhibit or arrest the further development of, the disease, condition, or disorder.

In certain embodiments, the particle is a non-naturally occurring particle.

In certain embodiments, the particle is selected from the group consisting of liposomes, virus-like particles, and lipid nanoparticles. The particle generally is roughly spherical in shape but it may take other shapes including, for example, polyhedrons, oblates, cylinders, boxes, cubes, cuboids, pyramids, and irregular three-dimensional shapes.

The particle generally is about 10 nm to about 2000 nm in its greatest diameter. In certain embodiments, the particle is about 10 nm to about 100 nm in its greatest diameter. In certain embodiments, the particle is about 10 nm to about 200 nm in its greatest diameter. In certain embodiments, the particle is about 10 nm to about 300 nm in its greatest diameter. In certain embodiments, the particle is about 10 nm to about 400 nm in its greatest diameter. In certain embodiments, the particle is about 10 nm to about 500 nm in its greatest diameter. In certain embodiments, the particle is about 10 nm to about 1000 nm in its greatest diameter. In certain embodiments, the particle is about 100 nm to about 200 nm in its greatest diameter. In certain embodiments, the particle is about 100 nm to about 300 nm in its greatest diameter. In certain embodiments, the particle is about 100 nm to about 400 nm in its greatest diameter. In certain embodiments, the particle is about 100 nm to about 500 nm in its greatest diameter. In certain embodiments, the particle is about 100 nm to about 1000 nm in its greatest diameter. In certain embodiments, the particle is about 100 nm to about 2000 nm in its greatest diameter. In certain embodiments, the particle is about 200 nm to about 500 nm in its greatest diameter. In certain embodiments, the particle is about 200 nm to about 1000 nm in its greatest diameter. In certain embodiments, the particle is about 200 nm to about 2000 nm in its greatest diameter. In certain embodiments, the particle is about 500 nm to about 1000 nm in its greatest diameter. In certain embodiments, the particle is about 500 nm to about 2000 nm in its greatest diameter. In certain embodiments, the particle is about 1000 nm to about 2000 nm in its greatest diameter.

The surface of the particle can be comprised of any biocompatible material, and, apart from the antigen, it can be homogeneous or heterogeneous in its composition.

In certain embodiments, the antigen is selected from the group consisting of viral antigens, bacterial antigens, tumor antigens, and tumor neoantigens. In certain embodiments, the antigen is a viral antigen. In certain embodiments, the antigen is a bacterial antigen. In certain embodiments, the antigen is a tumor antigen. In certain embodiments, the antigen is a tumor neoantigen. Examples of each these types of antigen are well known in the art.

The antigen forms or is physically associated with the surface of the particle. Typically, the antigen is in some way substantially anchored to the surface, for example through a covalent bond to a component of the surface. Alternatively or in addition, the antigen includes an anchoring domain which domain can be incorporated, covalently or noncovalently, into the general structure of the surface per se. In preferred embodiments, the physical association between the antigen and the surface of the particle is not mediated by an antigen-specific antibody or antigen-specific fragment thereof.

The interior of the particle comprises an immune modulating agent.

In certain embodiments, the immune modulating agent is substantially unassociated with the inner aspect or inner face of the surface. For example, in certain embodiments at least about 90 percent of the immune modulating agent is substantially unassociated with the inner aspect or inner face of the surface. In certain embodiments, the immune modulating agent is partially associated with the inner aspect or inner face of the surface. For example, in certain embodiments about 20 percent of the immune modulating agent is associated with the inner aspect or inner face of the surface, while the remainder of the immune modulating agent is unassociated with the inner aspect or inner face of the surface. In certain embodiments, the immune modulating agent is substantially associated with the inner aspect or inner face of the surface. For example, in certain embodiments at least about 90 percent of the immune modulating agent is associated with the inner aspect or inner face of the surface.

In certain embodiments, the immune modulating agent is not an expressable nucleic acid molecule, e.g., a gene.

In certain embodiments, the immune modulating agent is selected from the group consisting of therapeutic agents, immune activators, immune suppressors, and any combination thereof. In certain embodiments, the immune modulating agent is a therapeutic agent. In certain embodiments, the immune modulating agent is an immune activator. In certain embodiments, the immune modulating agent is an immune suppressor.

In certain embodiments, the immune modulating agent is a therapeutic agent. In certain embodiments, said therapeutic agent is selected from the group consisting of antisense, RNAi, aptamers, antagomirs, microRNAs, and any combination thereof. In certain embodiments, said therapeutic agent is antisense. In certain embodiments, said therapeutic agent is RNAi. In certain embodiments, said therapeutic agent is an aptamer. In certain embodiments, said therapeutic agent is an antagomir. In certain embodiments, said therapeutic agent is a microRNA.

In certain embodiments, the immune modulating agent is an immune activator.

In certain embodiments, the immune activator is a TLR agonist.

In certain embodiments, the TLR agonist is a TLR7 agonist.

In certain embodiments, the TLR agonist is a TLR9 agonist.

In certain embodiments, the TLR agonist is a synthetic CpG DNA oligonucleotide.

In certain embodiments, the immune modulating agent is a TLR antagonist.

In certain embodiments, the TLR antagonist is an S-class ODN.

In certain embodiments, the TLR antagonist is a TLR7 antagonist.

In certain embodiments, the TLR antagonist is a TLR9 antagonist.

In certain embodiments, the immune modulating agent is an immune activator, and the disease or condition is selected from the group consisting of cancer and infection.

In certain embodiments, the immune modulating agent is an immune activator, and the disease or condition is cancer. Cancers include cancer of skin, breast, lung, prostate, ovary, stomach, esophagus, colon, rectum, liver, pancreas, kidney, bladder, cervix, uterus, testis, brain, head and neck, eye, thyroid, muscle, and bone, as well as lymphoma, myeloma, and leukemia.

In certain embodiments, the immune modulating agent is an immune activator, and the disease or condition is infection. In certain embodiments, the immune modulating agent is an immune activator, and the infection is a viral infection. In certain embodiments, the immune modulating agent is an immune activator, and the infection is a bacterial infection.

In certain embodiments, the immune modulating agent is an immune activator, and the disease or condition is selected from the group consisting of primary tumors and metastatic tumors. In certain embodiments, the immune modulating agent is an immune activator, and the disease or condition is a primary tumor. In certain embodiments, the immune modulating agent is an immune activator, and the disease or condition is a metastatic tumor.

In certain embodiments, the immune modulating agent is an immune activator, and the disease or condition is selected from the group consisting of a primary liver tumor and a metastatic liver tumor. In certain embodiments, the immune modulating agent is an immune activator, and the disease or condition is a primary liver tumor. In certain embodiments, the immune modulating agent is an immune activator, and the disease or condition is a metastatic liver tumor.

In certain embodiments, the immune modulating agent is an immune activator, and the disease or condition is selected from the group consisting of a primary tumor in a lymph node and a metastatic tumor in a lymph node. In certain embodiments, the immune modulating agent is an immune activator, and the disease or condition is a primary tumor in a lymph node, i.e., a lymphoma. In certain embodiments, the immune modulating agent is an immune activator, and the disease or condition is a metastatic tumor in a lymph node, e.g., metastatic breast cancer.

In certain embodiments, the immune modulating agent is an immune activator, and the disease or condition is selected from the group consisting of a primary tumor in bone marrow and a metastatic tumor in bone marrow. In certain embodiments, the immune modulating agent is an immune activator, and the disease or condition is a primary tumor in bone marrow, e.g., multiple myeloma. In certain embodiments, the immune modulating agent is an immune activator and the disease or condition is a metastatic tumor in bone marrow, e.g., metastatic breast cancer.

In certain embodiments, the immune modulating agent is an immune activator, and the disease or condition is a viral hepatitis. In certain embodiments, the viral hepatitis is hepatitis A, i.e., the disease or condition characterized by the tissue expressing the first antigen is infection with HAV. In certain embodiments, the viral hepatitis is hepatitis B, i.e., the disease or condition characterized by the tissue expressing the first antigen is infection with HBV. In certain embodiments, the viral hepatitis is hepatitis C, i.e., the disease or condition characterized by the tissue expressing the first antigen is infection with HCV.

In certain embodiments, the immune modulating agent is an immune suppressor, and the disease or condition is selected from the group consisting of autoimmune diseases, allergy, and asthma. In certain embodiments, the immune modulating agent is an immune suppressor, and the disease or condition is an autoimmune disease. In certain embodiments, the immune modulating agent is an immune suppressor, and the disease or condition is allergy. In certain embodiments, the immune modulating agent is an immune suppressor, and the disease or condition is asthma.

In certain embodiments, the particle is administered intravenously.

In certain embodiments, the method further comprises administering to the subject an effective amount of a second therapeutic agent to treat the disease or condition.

In certain embodiments, the second therapeutic agent is selected from the group consisting of antisense, RNAi, aptamers, antagomirs, microRNAs, and any combination thereof. In certain embodiments, the second therapeutic agent is antisense. In certain embodiments, the second therapeutic agent is RNAi. In certain embodiments, the second therapeutic agent is an aptamer. In certain embodiments, the second therapeutic agent is an antagomir. In certain embodiments, the second therapeutic agent is a microRNA.

In certain embodiments, the second therapeutic agent is selected from the group consisting of TLR ligand, STING ligand, RIG-I ligand, cytokine, chemokine, checkpoint inhibitor, IDO inhibitor, anti-CD40 antibody, anti-OX40 antibody, anti-4-1BB antibody, NK cell activator, NK cell checkpoint inhibitor, and any combination thereof.

In certain embodiments, the second therapeutic agent is a TLR ligand.

In certain embodiments, the second therapeutic agent is a STING ligand.

In certain embodiments, the second therapeutic agent is a RIG-I ligand.

In certain embodiments, the second therapeutic agent is a cytokine.

In certain embodiments, the second therapeutic agent is a chemokine.

In certain embodiments, the second therapeutic agent is a checkpoint inhibitor, e.g., anti-CTLA-4, anti-PD-1, anti-PD-L1, anti-LAG-3, anti-TIM-3, anti-VISTA, or anti-GITR.

In certain embodiments, the second therapeutic agent is an IDO inhibitor.

In certain embodiments, the second therapeutic agent is an anti-CD40 antibody.

In certain embodiments, the therapeutic agent for treating the disease or condition characterized by the first antigen is an anti-OX40 antibody.

In certain embodiments, the second therapeutic agent is an anti-4-1BB antibody.

In certain embodiments, the second therapeutic agent is an NK cell activator.

In certain embodiments, the second therapeutic agent is an NK cell checkpoint inhibitor.

In certain embodiments, the second therapeutic agent is administered systemically.

In certain embodiments, the second therapeutic agent is administered locally.

In certain embodiments, the disease or condition is a tumor; and the second therapeutic agent is administered intratumorally.

In certain embodiments, the subject is a human.

An aspect of the invention is a method of treating a disease or condition. The method includes the steps of:

immunogenically administering to a subject having a disease or condition an effective amount of a first particle comprising a surface and an interior, wherein the surface of the first particle comprises an antigen, and the interior of the first particle optionally comprises a first immune modulating agent, to immunize the subject against the antigen; and

systemically administering to the subject an effective amount of a second particle comprising a surface and an interior, wherein the surface of the second particle comprises the antigen, and the interior of the second particle comprises a second immune modulating agent, to modulate an immune response of the subject,

thereby treating the disease or condition.

In certain embodiments, the first particle is a non-naturally occurring particle.

In certain embodiments, the first particle is selected from the group consisting of liposomes, virus-like first particles, and lipid nanoparticles. The first particle generally is roughly spherical in shape but it may take other shapes including, for example, polyhedrons, oblates, cylinders, boxes, cubes, cuboids, pyramids, and irregular three-dimensional shapes.

The first particle generally is about 10 nm to about 2000 nm in its greatest diameter. In certain embodiments, the first particle is about 10 nm to about 100 nm in its greatest diameter. In certain embodiments, the first particle is about 10 nm to about 200 nm in its greatest diameter. In certain embodiments, the first particle is about 10 nm to about 300 nm in its greatest diameter. In certain embodiments, the first particle is about 10 nm to about 400 nm in its greatest diameter. In certain embodiments, the first particle is about 10 nm to about 500 nm in its greatest diameter. In certain embodiments, the first particle is about 10 nm to about 1000 nm in its greatest diameter. In certain embodiments, the first particle is about 100 nm to about 200 nm in its greatest diameter. In certain embodiments, the first particle is about 100 nm to about 300 nm in its greatest diameter. In certain embodiments, the first particle is about 100 nm to about 400 nm in its greatest diameter. In certain embodiments, the first particle is about 100 nm to about 500 nm in its greatest diameter. In certain embodiments, the first particle is about 100 nm to about 1000 nm in its greatest diameter. In certain embodiments, the first particle is about 100 nm to about 2000 nm in its greatest diameter. In certain embodiments, the first particle is about 200 nm to about 500 nm in its greatest diameter. In certain embodiments, the first particle is about 200 nm to about 1000 nm in its greatest diameter. In certain embodiments, the first particle is about 200 nm to about 2000 nm in its greatest diameter. In certain embodiments, the first particle is about 500 nm to about 1000 nm in its greatest diameter. In certain embodiments, the first particle is about 500 nm to about 2000 nm in its greatest diameter. In certain embodiments, the first particle is about 1000 nm to about 2000 nm in its greatest diameter.

The surface of the first particle can be comprised of any biocompatible material, and, apart from the antigen, it can be homogeneous or heterogeneous in its composition.

In certain embodiments, the antigen is selected from the group consisting of viral antigens, bacterial antigens, tumor antigens, and tumor neoantigens. In certain embodiments, the antigen is a viral antigen. In certain embodiments, the antigen is a bacterial antigen. In certain embodiments, the antigen is a tumor antigen. In certain embodiments, the antigen is a tumor neoantigen. Examples of each these types of antigen are well known in the art.

The antigen forms or is physically associated with the surface of the first particle. Typically, the antigen is in some way substantially anchored to the surface of the first particle, for example through a covalent bond to a component of the surface of the first particle. Alternatively or in addition, the antigen includes an anchoring domain which domain can be incorporated, covalently or noncovalently, into the general structure of the surface per se of the first particle. In preferred embodiments, the physical association between the antigen and the surface of the first particle is not mediated by an antigen-specific antibody or antigen-specific fragment thereof.

In certain embodiments, the first interior of the first particle comprises a first immune modulating agent.

In certain embodiments, the first immune modulating agent is substantially unassociated with the inner aspect or inner face of the surface of the first particle. For example, in certain embodiments at least about 90 percent of the first immune modulating agent is substantially unassociated with the inner aspect or inner face of the surface of the first particle. In certain embodiments, the first immune modulating agent is partially associated with the inner aspect or inner face of the surface of the first particle. For example, in certain embodiments about 20 percent of the first immune modulating agent is associated with the inner aspect or inner face of the surface of the first particle, while the remainder of the first immune modulating agent is unassociated with the inner aspect or inner face of the surface of the first particle. In certain embodiments, the first immune modulating agent is substantially associated with the inner aspect or inner face of the surface of the first particle. For example, in certain embodiments at least about 90 percent of the first immune modulating agent is associated with the inner aspect or inner face of the surface of the first particle.

In certain embodiments, the first immune modulating agent is not an expressable nucleic acid molecule, e.g., a gene.

In certain embodiments, the first immune modulating agent is selected from the group consisting of therapeutic agents, immune activators, and any combination thereof. In certain embodiments, the first immune modulating agent is a therapeutic agent. In certain embodiments, the first immune modulating agent is an immune activator.

In certain embodiments, the first immune modulating agent is a therapeutic agent, and said therapeutic agent is selected from the group consisting of antisense, RNAi, aptamers, antagomirs, microRNAs, and any combination thereof. In certain embodiments, said therapeutic agent is antisense. In certain embodiments, said therapeutic agent is RNAi. In certain embodiments, said therapeutic agent is an aptamer. In certain embodiments, said therapeutic agent is an antagomir. In certain embodiments, said therapeutic agent is a microRNA.

In certain embodiments, the first immune modulating agent is an immune activator. In certain embodiments, said immune activator is a TLR agonist. In certain embodiments, said TLR agonist is a TLR7 agonist. In certain embodiments, said TLR agonist is a TLR9 agonist. In certain embodiments, said TLR agonist is a synthetic CpG DNA oligonucleotide.

In certain embodiments, the second particle is a non-naturally occurring particle.

In certain embodiments, the second particle is selected from the group consisting of liposomes, virus-like second particles, and lipid nanoparticles. The second particle generally is roughly spherical in shape but it may take other shapes including, for example, polyhedrons, oblates, cylinders, boxes, cubes, cuboids, pyramids, and irregular three-dimensional shapes.

The second particle generally is about 10 nm to about 2000 nm in its greatest diameter. In certain embodiments, the second particle is about 10 nm to about 100 nm in its greatest diameter. In certain embodiments, the second particle is about 10 nm to about 200 nm in its greatest diameter. In certain embodiments, the second particle is about 10 nm to about 300 nm in its greatest diameter. In certain embodiments, the second particle is about 10 nm to about 400 nm in its greatest diameter. In certain embodiments, the second particle is about 10 nm to about 500 nm in its greatest diameter. In certain embodiments, the second particle is about 10 nm to about 1000 nm in its greatest diameter. In certain embodiments, the second particle is about 100 nm to about 200 nm in its greatest diameter. In certain embodiments, the second particle is about 100 nm to about 300 nm in its greatest diameter. In certain embodiments, the second particle is about 100 nm to about 400 nm in its greatest diameter. In certain embodiments, the second particle is about 100 nm to about 500 nm in its greatest diameter. In certain embodiments, the second particle is about 100 nm to about 1000 nm in its greatest diameter. In certain embodiments, the second particle is about 100 nm to about 2000 nm in its greatest diameter. In certain embodiments, the second particle is about 200 nm to about 500 nm in its greatest diameter. In certain embodiments, the second particle is about 200 nm to about 1000 nm in its greatest diameter. In certain embodiments, the second particle is about 200 nm to about 2000 nm in its greatest diameter. In certain embodiments, the second particle is about 500 nm to about 1000 nm in its greatest diameter. In certain embodiments, the second particle is about 500 nm to about 2000 nm in its greatest diameter. In certain embodiments, the second particle is about 1000 nm to about 2000 nm in its greatest diameter.

The surface of the second particle can be comprised of any biocompatible material, and, apart from the antigen, it can be homogeneous or heterogeneous in its composition.

The antigen forms or is physically associated with the surface of the second particle. Typically, the antigen is in some way substantially anchored to the surface of the second particle, for example through a covalent bond to a component of the surface of the second particle. Alternatively or in addition, the antigen includes an anchoring domain which domain can be incorporated, covalently or noncovalently, into the general structure of the surface per se of the second particle. In preferred embodiments, the physical association between the antigen and the surface of the second particle is not mediated by an antigen-specific antibody or antigen-specific fragment thereof.

The interior of the second particle comprises a second immune modulating agent.

In certain embodiments, the second immune modulating agent is substantially unassociated with the inner aspect or inner face of the surface of the second particle. For example, in certain embodiments at least about 90 percent of the second immune modulating agent is substantially unassociated with the inner aspect or inner face of the surface of the second particle. In certain embodiments, the second immune modulating agent is partially associated with the inner aspect or inner face of the surface of the second particle. For example, in certain embodiments about 20 percent of the second immune modulating agent is associated with the inner aspect or inner face of the surface of the second particle, while the remainder of the second immune modulating agent is unassociated with the inner aspect or inner face of the surface of the second particle. In certain embodiments, the second immune modulating agent is substantially associated with the inner aspect or inner face of the surface of the second particle. For example, in certain embodiments at least about 90 percent of the second immune modulating agent is associated with the inner aspect or inner face of the surface of the second particle.

In certain embodiments, the second immune modulating agent is not an expressable nucleic acid molecule, e.g., a gene.

In certain embodiments, the second immune modulating agent is selected from the group consisting of therapeutic agents, immune activators, immune suppressors, and any combination thereof. In certain embodiments, the second immune modulating agent is a therapeutic agent. In certain embodiments, the second immune modulating agent is an immune activator. In certain embodiments, the second immune modulating agent is an immune suppressor.

In certain embodiments, the second immune modulating agent is a therapeutic agent, and said therapeutic agent is selected from the group consisting of antisense, RNAi, aptamers, antagomirs, microRNAs, and any combination thereof. In certain embodiments, said therapeutic agent is antisense. In certain embodiments, said therapeutic agent is RNAi. In certain embodiments, said therapeutic agent is an aptamer. In certain embodiments, said therapeutic agent is an antagomir. In certain embodiments, said therapeutic agent is a microRNA.

In certain embodiments, the second immune modulating agent is an immune activator. In certain embodiments, said immune activator is a TLR agonist. In certain embodiments, said TLR agonist is a TLR7 agonist. In certain embodiments, said TLR agonist is a TLR9 agonist. In certain embodiments, said TLR agonist is a synthetic CpG DNA oligonucleotide.

In certain embodiments, the second immune modulating agent is an immune suppressor.

In certain embodiments, the second immune modulating agent is a TLR antagonist.

In certain embodiments, the second immune modulating agent is an S-class ODN.

In certain embodiments, the second immune modulating agent is a TLR7 antagonist.

In certain embodiments, the second immune modulating agent is a TLR9 antagonist.

In certain embodiments, the first particle and the second particle are the same.

In certain embodiments, the first particle and the second particle are different.

In certain embodiments, the first immune modulating agent and the second immune modulating agent are the same.

In certain embodiments, the first immune modulating agent and the second immune modulating agent are different.

In certain embodiments, the second immune modulating agent is an immune activator, and the disease or condition is selected from the group consisting of cancer and infection.

In certain embodiments, the second immune modulating agent is an immune activator, and the disease or condition is cancer.

In certain embodiments, the second immune modulating agent is an immune activator, and the disease or condition is infection. In certain embodiments, the second immune modulating agent is an immune activator, and the infection is a viral infection. In certain embodiments, the second immune modulating agent is an immune activator, and the infection is a bacterial infection.

In certain embodiments, the second immune modulating agent is an immune activator, and the disease or condition is selected from the group consisting of primary tumors and metastatic tumors. In certain embodiments, the second immune modulating agent is an immune activator, and the disease or condition is a primary tumor. In certain embodiments, the second immune modulating agent is an immune activator, and the disease or condition is a metastatic tumor.

In certain embodiments, the second immune modulating agent is an immune activator, and the disease or condition is selected from the group consisting of a primary liver tumor and a metastatic liver tumor. In certain embodiments, the second immune modulating agent is an immune activator, and the disease or condition is a primary liver tumor. In certain embodiments, the second immune modulating agent is an immune activator, and the disease or condition is a metastatic liver tumor.

In certain embodiments, the second immune modulating agent is an immune activator, and the disease or condition is selected from the group consisting of a primary tumor in a lymph node and a metastatic tumor in a lymph node. In certain embodiments, the second immune modulating agent is an immune activator, and the disease or condition is a primary tumor in a lymph node, i.e., a lymphoma. In certain embodiments, the second immune modulating agent is an immune activator, and the disease or condition is a metastatic tumor in a lymph node, e.g., metastatic breast cancer.

In certain embodiments, the second immune modulating agent is an immune activator, and the disease or condition is selected from the group consisting of a primary tumor in bone marrow and a metastatic tumor in bone marrow. In certain embodiments, the second immune modulating agent is an immune activator, and the disease or condition is a primary tumor in bone marrow, e.g., multiple myeloma. In certain embodiments, the second immune modulating agent is an immune activator and the disease or condition is a metastatic tumor in bone marrow, e.g., metastatic breast cancer.

In certain embodiments, the second immune modulating agent is an immune activator, and the disease or condition is a viral hepatitis. In certain embodiments, the viral hepatitis is hepatitis A, i.e., the disease or condition characterized by the tissue expressing the first antigen is infection with HAV. In certain embodiments, the viral hepatitis is hepatitis B, i.e., the disease or condition characterized by the tissue expressing the first antigen is infection with HBV. In certain embodiments, the viral hepatitis is hepatitis C, i.e., the disease or condition characterized by the tissue expressing the first antigen is infection with HCV.

In certain embodiments, the second immune modulating agent is an immune suppressor, and the disease or condition is selected from the group consisting of autoimmune diseases, allergy, and asthma. In certain embodiments, the second immune modulating agent is an immune suppressor, and the disease or condition is an autoimmune disease. In certain embodiments, the second immune modulating agent is an immune suppressor, and the disease or condition is allergy. In certain embodiments, the second immune modulating agent is an immune suppressor, and the disease or condition is asthma.

In certain embodiments, the second particle is administered intravenously.

In certain embodiments, the method further comprises administering to the subject an effective amount of a second therapeutic agent to treat the disease or condition.

In certain embodiments, the second therapeutic agent is selected from the group consisting of antisense, RNAi, aptamers, antagomirs, microRNAs, and any combination thereof. In certain embodiments, the second therapeutic agent is antisense. In certain embodiments, the second therapeutic agent is RNAi. In certain embodiments, the second therapeutic agent is an aptamer. In certain embodiments, the second therapeutic agent is an antagomir. In certain embodiments, the second therapeutic agent is a microRNA.

In certain embodiments, the second therapeutic agent is selected from the group consisting of TLR ligand, STING ligand, RIG-I ligand, cytokine, chemokine, checkpoint inhibitor, IDO inhibitor, anti-CD40 antibody, anti-OX40 antibody, anti-4-1BB antibody, NK cell activator, NK cell checkpoint inhibitor, and any combination thereof.

In certain embodiments, the second therapeutic agent is a TLR ligand.

In certain embodiments, the second therapeutic agent is a STING ligand.

In certain embodiments, the second therapeutic agent is a RIG-I ligand.

In certain embodiments, the second therapeutic agent is a cytokine.

In certain embodiments, the second therapeutic agent is a chemokine.

In certain embodiments, the second therapeutic agent is a checkpoint inhibitor, e.g., anti-CTLA-4, anti-PD-1, anti-PD-L1, anti-LAG-3, anti-TIM-3, anti-VISTA, or anti-GITR.

In certain embodiments, the second therapeutic agent is an IDO inhibitor.

In certain embodiments, the second therapeutic agent is an anti-CD40 antibody.

In certain embodiments, the therapeutic agent for treating the disease or condition characterized by the first antigen is an anti-OX40 antibody.

In certain embodiments, the second therapeutic agent is an anti-4-1BB antibody.

In certain embodiments, the second therapeutic agent is an NK cell activator.

In certain embodiments, the second therapeutic agent is an NK cell checkpoint inhibitor.

In certain embodiments, the second therapeutic agent is administered systemically.

In certain embodiments, the second therapeutic agent is administered locally.

In certain embodiments, the disease or condition is a tumor; and the second therapeutic agent is administered intratumorally.

In certain embodiments, the subject is a human.

### Dosing

Dosage regimens can be adjusted to provide the optimum desired response. For example, a single bolus can be administered, several divided doses can be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present invention. Further, one skilled in the art would understand, once armed with the teachings provided herein, that a therapeutic benefit, such as, but not limited to, detectable decrease in tumor size and/or metastasis, and increased time to recurrence, among many other parameters, can be assessed by a wide variety of methods known in the art for assessing the efficacy of treatment of cancer, and these methods are encompassed herein, as well as methods to be developed in the future.

Following the initial priming dose of the particle through an immunogenic route of administration, a subject will make an antibody response to the antigen (that is on the surface of the particle). When such a subject is then administered a particle through a therapeutic route such as IV, circulating antibodies will bind to the antigen on the surface of the particle in the circulation, forming circulating immune complexes (CIC). The general principles governing the formation and immune effects and potential toxicity of circulating immune complexes are well understood among persons skilled in the art (e.g., Theofilopoulos, A. N. et al. (1980) “Immune complexes in human diseases: a review.” Am J Pathol 100(2):529-94; Schifferli, J. A. et al. (1989) “Physiological and pathological aspects of circulating immune complexes.” Kidney Int 35(4):993-1003). Briefly, an important concept relevant to the current invention is that immune complexes which form under conditions of antibody excess tend to be large and insoluble and are removed from the circulation by FcR-expressing cells without depositing extensively in tissues, and thus without causing toxicity, while immune complexes that form under conditions closer to antigen:antibody equivalence can be of intermediate size, and circulate broadly, often depositing in tissues such as the kidney and joints, leading to inflammatory reactions and toxicity. Theofilopoulos, A. N. et al. (1980) “Immune complexes in human diseases: a review.” Am J Pathol 100(2):529-94. To avoid such toxicity, the current invention features methods to administer the therapeutic IV doses of the particles by a slow infusion such that sudden large doses/concentrations of particles (that might form large immune complexes due to a local high antigen concentration) are avoided. Such infusions may be given over 5 minutes, 30 minutes, an hour, or several or many hours, or by a continuous infusion using a medical device such as an infusion pump, which may be worn by the patient. For safety the particle infusion may be started at a very slow rate and the subject's response monitored clinically during the infusion. If the subject is tolerating the infusion without clinical signs of immune complex formation or particle toxicity (such as typical infusion reactions with fever, chills, back pain, headache, nausea, muscle pain, joint pain, rash, etc.), then the infusion rate may be increased at time intervals of 5 min, 15 min, or 30 min. Conversely, if clinical toxicity is seen during the infusion, then the rate can be slowed or stopped.

Preferred particles and antigens of the present invention are highly immunogenic and are Th1-biased, or at least are not Th2-biased, so that little or no IgE antibodies are made to the antigen, and so that the initial priming exposure induces a strong IgG antibody response that will facilitate particle uptake via the desired FcR. A high initial serum level of antibody to the antigen makes it possible to administer a greater therapeutic dose of the particles in less time during the infusion without toxicity, because the immune complexes form under conditions of great Ab excess. Theofilopoulos, A. N. et al. (1980) “Immune complexes in human diseases: a review.” Am J Pathol 100(2):529-94.

In some embodiments of the invention, a test is performed to determine the subject's serum level of antibody to the antigen prior to the therapeutic infusion, and the dose or rate of the infusion can be adjusted based on the amount of serum antibody.

For adult human subjects, priming (immunizing) doses of the particles described herein typically range from about 1 mg/dose to about 100 mg/dose, more typically from about 5 mg/dose to about 40 mg/dose, and most typically from about 10 mg/dose to about 20 mg/dose. Doses will depend on factors including the route of administration, e.g., oral administration may require a substantially larger dose than subcutaneous or other parenteral administration.

Following the priming dose of the particle, the therapeutic IV dose may be administered at any timepoint after the subject has generated a strong Ab response to the antigen, for as long as the response is maintained in the serum. Using a preferred immunogenic particle this Ab response is induced within one week of the priming injection, at which time the therapeutic IV infusion may be administered as described above. Doses and infusion rates for the therapeutic infusion may depend on the strength of the antibody response to the second antigen, but with preferred particles will typically range from about 10 mg/dose to about 10,000 mg/dose, more typically from about 50 mg/dose to about 1,000 mg/dose, and most typically from about 100 mg/dose to about 500 mg/dose.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the active compound or compounds are well-known in the relevant art and would be understood to be encompassed by the skilled artisan once provided the teachings disclosed herein.

### Formulation

In the discussion that follows, “active agent” can refer to any particle, therapeutic, or other agent to be administered to a subject in accordance with any method of the invention.

An active agent can be combined with a pharmaceutically acceptable carrier to provide a pharmaceutical composition.

As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, trehalose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the active agent.

The active agent or other therapeutics may be administered alone (e.g., in saline or buffer) or using any delivery vehicles known in the art.

Combined with the teachings provided herein, by choosing among the various active agents and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular active agent being administered, the size of the subject, or the severity of the disease or condition. One of ordinary skill in the art can empirically determine the effective amount of a particular active agent and/or other therapeutic agent without necessitating undue experimentation.

The active agents of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The active agent can be given in conjunction with other agents known in the art to be useful in combination with other agents known to be useful in the treatment of anxiety or depression.

For use in therapy, an effective amount of the active agent can be administered to a subject by any mode that delivers the active agent to the desired site, e.g., mucosal, systemic. “Administering” the active agent or pharmaceutical composition of the present invention may be accomplished by any means known to the skilled artisan. Preferred routes of administration include but are not limited to oral, subcutaneous, intramuscular, intranasal, intratracheal, inhalational, and rectal.

For oral administration, the agents (i.e., active agent or agents) can be formulated readily by combining the active agent(s) with pharmaceutically acceptable carriers well known in the art. Such carriers enable the agents of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agent doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner

For administration by inhalation, the agents for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the agent and a suitable powder base such as lactose or starch.

The agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active agents in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the agents to allow for the preparation of highly concentrated solutions.

Alternatively, the active agents may be in powder form for constitution with a suitable vehicle, e.g., sterile pyrogen-free water, before use.

The agents may also be formulated in rectal or vaginal compositions such as suppositories or retention enemas, e.g., containing conventional suppository bases such as cocoa butter or other glycerides.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active agents, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer Science 249:1527 (1990), which is incorporated herein by reference.

The active agent(s) may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulfuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2 percent w/v); citric acid and a salt (1-3 percent w/v); boric acid and a salt (0.5-2.5 percent w/v); and phosphoric acid and a salt (0.8-2 percent w/v). Suitable preservatives include benzalkonium chloride (0.003-0.03 percent w/v); chlorobutanol (0.3-0.9 percent w/v); parabens (0.01-0.25 percent w/v) and thimerosal (0.004-0.02 percent w/v).

The pharmaceutical compositions of the invention contain an effective amount of an active agent and optionally other therapeutic agents optionally included in a pharmaceutically-acceptable carrier. The term “pharmaceutically-acceptable carrier” means one or more compatible solid or liquid filler, diluants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the agents of the present invention, and with each other, in a manner such that there is no interaction which would substantially impair the desired pharmaceutical efficiency.

A variety of administration routes are available. The particular mode selected will depend, of course, upon the particular active agent selected, the particular condition being treated, and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed above.

The compositions may conveniently be presented in unit dosage form and may be prepared by any of the methods well known in the art of pharmacy. All methods include the step of bringing the agents into association with a carrier which constitutes one or more accessory ingredients. In general, the compositions are prepared by uniformly and intimately bringing the agents into association with a liquid carrier, a finely divided solid carrier, or both, and then, if necessary, shaping the product. Liquid dose units are vials or ampoules. Solid dose units are tablets, capsules and suppositories. For treatment of a patient, depending on activity of the agent, manner of administration, purpose of the administration (i.e., prophylactic or therapeutic), nature and severity of the disorder, age and body weight of the patient, different doses may be necessary. The administration of a given dose can be carried out both by single administration in the form of an individual dose unit or else several smaller dose units.

Other delivery systems can include time-release, delayed release or sustained release delivery systems. Such systems can avoid repeated administrations of the agents, increasing convenience to the subject and the physician. Many types of release delivery systems are available and known to those of ordinary skill in the art. They include polymer-based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Having now described the present invention in detail, the same will be more clearly understood by reference to the following examples, which are included herewith for purposes of illustration only and are not intended to be limiting of the invention.

### EXAMPLES

### Example 1. Tolerability of CMP-001 Slow IV Infusion in Primed Healthy Mice

In a preliminary study using mice having anti-Qb antibodies from previous CMP-001 exposure, it was found that such mice did not tolerate IV bolus administration of 100 μg CMP-001 (i.e., 100 μg G10 CpG oligodeoxynucleotide (SEQ ID NO: 32) formulated with 400 μg Qb (VLP)). These mice had been primed in advance (D-19) with 12.5 μg CMP-001 administered subcutaneously.

A single-administration tolerability study was then undertaken to investigate whether slow IV infusion administration of 100 μg CMP-001 (i.e., 100 μg G10 CpG oligodeoxynucleotide (SEQ ID NO: 32) formulated with 400 μg Qb (VLP)) could eliminate toxicity observed with IV bolus in mice with anti-Qb antibodies from previous CMP-001 exposure.

TABLE 1
Study design for tolerability study.

| | | | | | | Treatment |
|---|---|---|---|---|---|---|
| Group | N | Prime¹ | Treatment | Dose | Route | schedule |
| 1 | 8 | Yes | CMP-001 | 100 μg in | 1 hr slow IV | ×1 |
| | | | | 100 μL | infusion | |
| 2 | 8 | Yes | CMP-001 | 100 μg in | 30 min slow | ×1 |
| | | | | 100 μL | IV infusion | |
| 3 | 8 | Yes | CMP-001 | 100 μg in | 15 min slow | ×1 |
| | | | | 100 μL | IV infusion | |

¹Prime: 12.5 μg/100 μL CMP-001 SC at D-14

The results from this study demonstrated that slow infusion enables IV administration of CMP-001 in animals with pre-existing anti-Qb antibodies. Infusion times of 60 minutes and 30 minutes were well tolerated. An infusion time of 15 minutes was tolerated, but signs of stress were noted with this shortest duration infusion. See Table 2 and Table 3.

TABLE 2
Body weight monitoring following varied duration IV infusion of CMP-001 in mice with pre-existing
anti-Qb antibodies.

| | | | | | | | | | D19 | D19 | D20 | D20 |
|---|---|---|---|---|---|---|---|---|---|---|---|---|
| | ID | D0 | D3 | D6 | D11 | D14 | D17 | D18 | pre | 12 h post | pre | 12 h post |
| Group 1 | 54320 | 19.3 | 19.9 | 20.3 | 20.0 | 21.4 | 21.2 | 21.2 | 22.2 | 20.18 | 19.2 | |
| | 54133 | 16.5 | 17.7 | 17.8 | 18.0 | 18.4 | 19.2 | 19.1 | 19.5 | 17.90 | 17.1 | |
| | 54457 | 19.9 | 19.8 | 20.4 | 20.5 | 21.5 | 21.3 | 22.5 | 22.2 | 21.20 | 19.9 | |
| | 54317 | 19.1 | 20.0 | 19.9 | 19.8 | 20.5 | 21.5 | 21.3 | 22.0 | 20.36 | 19.4 | |
| | 54314 | 19.3 | 19.8 | 20.9 | 19.5 | 20.1 | 19.9 | 20.2 | 20.6 | 19.34 | 18.6 | |
| | 54090 | 20.4 | 20.3 | 20.8 | 20.5 | 21.7 | 21.2 | 21.8 | 22.9 | 20.90 | 19.7 | |
| | 54136 | 18.6 | 19.4 | 20.0 | 20.4 | 20.4 | 20.7 | 20.4 | 21.2 | 19.30 | 18.6 | |
| | 54490 | 19.9 | 19.7 | 19.8 | 20.2 | 21.6 | 20.6 | 21.8 | 22.3 | 20.78 | 19.4 | |
| | Mean | 19.1 | 19.6 | 20.0 | 19.9 | 20.7 | 20.7 | 21.1 | 21.6 | 20.00 | 19.0 | |
| | SD | 1.2 | 0.8 | 1.0 | 0.8 | 1.1 | 0.8 | 1.1 | 1.1 | 1.09 | 0.9 | |
| Group 2 | 54313 | 19.2 | 20.4 | 20.8 | 20.6 | 21.0 | 21.4 | 21.5 | 21.8 | | 21.8 | 21.17 |
| | 54401 | 17.7 | 18.6 | 18.8 | 18.9 | 19.4 | 19.5 | 20.2 | 20.2 | | 20.0 | 18.66 |
| | 54478 | 17.3 | 18.3 | 18.9 | 18.2 | 18.8 | 18.6 | 19.3 | 19.4 | | 19.4 | 19.39 |
| | 54177 | 19.3 | 19.9 | 20.3 | 20.3 | 22.1 | 21.5 | 22.6 | | | 21.6 | 20.85 |
| | 54199 | 18.8 | 19.8 | 20.3 | 20.4 | 21.7 | 21.3 | 21.9 | 22.5 | | 22.0 | 20.94 |
| | 54102 | 19.0 | 19.5 | 19.7 | 20.1 | 21.0 | 20.4 | 21.0 | 21.6 | | 20.8 | 19.76 |
| | 54057 | 20.2 | 20.2 | 21.3 | 20.8 | 22.3 | 21.7 | 22.3 | 22.5 | | 22.4 | 20.68 |
| | 54181 | 19.5 | 20.0 | 20.0 | 20.0 | 19.9 | 20.8 | 21.3 | 20.8 | | 21.2 | 20.16 |
| | Mean | 18.9 | 19.6 | 20.0 | 19.9 | 20.8 | 20.7 | 21.3 | 21.2 | | 21.2 | 20.20 |
| | SD | 1.0 | 0.8 | 0.8 | 0.9 | 1.3 | 1.1 | 1.1 | 1.2 | | 1.0 | 0.88 |
| Group 3 | 50398 | 20.0 | 20.3 | 20.5 | 20.4 | 21.1 | 20.7 | 21.0 | 21.4 | | 21.1 | |
| | 54079 | 18.0 | 17.5 | 18.2 | 18.3 | 19.4 | 19.8 | 20.1 | 20.3 | | 19.3 | |
| | 54296 | 18.4 | 18.9 | 19.6 | 19.6 | 19.8 | 20.0 | 20.1 | 20.7 | | 20.2 | |
| | 54341 | 19.7 | 20.8 | 21.2 | 21.1 | 21.9 | 22.1 | 22.2 | 22.9 | | 22.6 | |
| | 54455 | 19.7 | 21.2 | 21.6 | 21.7 | 21.9 | 22.5 | 22.3 | 23.1 | | 22.6 | |
| | 54235 | 18.7 | 19.8 | 20.1 | 20.4 | 21.6 | 21.3 | 21.5 | 22.2 | | 21.4 | |
| | 54200 | 19.6 | 19.8 | 21.0 | 20.5 | 21.0 | 20.6 | 20.9 | 21.7 | | 21.2 | |
| | 57678 | 19.4 | 19.6 | 20.9 | 21.1 | 21.9 | 21.3 | 21.4 | 22.2 | | 21.7 | |
| | Mean | 19.2 | 19.7 | 20.4 | 20.4 | 21.1 | 21.0 | 21.2 | 21.8 | | 21.2 | |
| | SD | 0.7 | 1.2 | 1.1 | 1.0 | 1.0 | 0.9 | 0.8 | 1.0 | | 1.1 | |

| | | D21 | D21 | | | | BW Δ % | BW Δ % | BW Δ % |
|---|---|---|---|---|---|---|---|---|---|
| | ID | pre | 12 h post | D22 | D24 | D27 | D27-D0 | D27-D18 | D27-D24 |
| Group 1 | 54320 | 20.3 | | 22.1 | 21.4 | 22.3 | 15.7 | 5.0 | 4.4 |
| | 54133 | 18.8 | | 19.5 | 19.0 | 19.6 | 18.7 | 2.6 | 3.4 |
| | 54457 | 21.0 | | 22.2 | 21.6 | 22.3 | 12.2 | −0.8 | 3.2 |
| | 54317 | 20.6 | | 22.0 | 21.8 | 22.3 | 17.0 | 5.0 | 2.4 |
| | 54314 | 19.9 | | 20.7 | 20.5 | 21.3 | 10.4 | 5.4 | 3.9 |
| | 54090 | 20.6 | | 22.1 | 21.8 | 22.7 | 11.1 | 3.9 | 4.1 |
| | 54136 | 20.1 | | 21.5 | 20.9 | 21.8 | 17.3 | 6.6 | 4.4 |
| | 54490 | 20.8 | | 22.1 | 21.3 | 23.0 | 16.1 | 5.7 | 8.1 |
| | Mean | 20.2 | | 21.5 | 21.0 | 21.9 | | | |
| | SD | 0.7 | | 1.0 | 0.9 | 1.1 | | | |
| Group 2 | 54313 | 20.7 | | 21.7 | 21.7 | 21.8 | 13.9 | 1.6 | 0.8 |
| | 54401 | 18.4 | | 19.5 | 20.7 | 20.6 | 16.1 | 2.0 | −0.7 |
| | 54478 | 18.8 | | 19.5 | 20.0 | 19.7 | 14.0 | 1.8 | −1.5 |
| | 54177 | 20.0 | | 21.2 | 22.2 | 22.2 | 15.3 | −1.7 | −0.1 |
| | 54199 | 20.2 | | 20.9 | 22.0 | 21.8 | 15.9 | −0.3 | −0.8 |
| | 54102 | 19.1 | | 20.0 | 21.7 | 21.3 | 12.2 | 1.4 | −1.5 |
| | 54057 | 19.7 | | 21.0 | 22.3 | 21.8 | 8.0 | −2.3 | −2.1 |
| | 54181 | 19.1 | | 19.6 | 21.0 | 21.2 | 8.8 | −0.3 | 1.2 |
| | Mean | 19.5 | | 20.4 | 21.4 | 21.3 | | | |
| | SD | 0.8 | | 0.9 | 0.8 | 0.8 | | | |
| Group 3 | 50398 | 21.4 | 20.53 | 19.8 | 21.3 | 21.8 | 9.5 | 4.1 | 2.4 |
| | 54079 | 19.9 | 19.43 | 19.4 | 19.4 | 20.0 | 10.8 | −0.5 | 3.1 |
| | 54296 | 20.9 | 20.07 | 19.9 | 20.6 | 21.4 | 16.4 | 6.3 | 3.8 |
| | 54341 | 22.6 | 21.54 | 21.0 | 22.7 | 22.7 | 15.2 | 2.3 | 0.1 |
| | 54455 | 23.1 | 21.65 | 21.0 | 22.7 | 23.0 | 17.3 | 3.6 | 1.7 |
| | 54235 | 21.2 | 20.24 | 19.5 | 20.9 | 21.2 | 13.1 | −1.3 | 1.3 |
| | 54200 | 21.5 | 20.91 | 20.0 | 21.3 | 21.3 | 9.1 | 2.3 | 0.2 |
| | 57678 | 22.4 | 21.21 | 20.9 | 22.0 | 22.4 | 15.7 | 4.5 | 1.8 |
| | Mean | 21.6 | 20.70 | 20.2 | 21.4 | 21.7 | | | |
| | SD | 1.0 | 0.77 | 0.7 | 1.1 | 1.0 | | | |

Group 1: CMP-001 100 μg IV 1 hour D0
Group 2: CMP-001 100 μg IV 30 min D1
Group 3: CMP-001 100 μg IV 15 D2

TABLE 3
Observations of animals following varied duration IV infusion of CMP-001 in mice with pre-existing anti-Qb antibodies.

| | | BW (g) | Observations | BW (g) | % BW Δ | Observations | BW (g) | % BW Δ | Observations |
|---|---|---|---|---|---|---|---|---|---|
| Group | ID | pre | post | 12 h post | 12 h post | 12 h post | 24 h post | 24 h post | 24 h post |
| 1 | 54320 | 22.2 | nothing to report | 20.2 | −8.9 | nothing to report | 19.2 | −13.4 | nothing to report |
| | 54133 | 19.5 | | 17.9 | −8.1 | | 17.1 | −12.0 | |
| | 54457 | 22.2 | | 21.2 | −4.4 | | 19.9 | −10.3 | |
| | 54317 | 22.0 | | 20.4 | −7.6 | | 19.4 | −12.2 | |
| | 54314 | 20.6 | | 19.3 | −6.3 | | 18.6 | −9.7 | |
| | 54090 | 22.9 | | 20.9 | −8.6 | | 19.7 | −13.8 | |
| | 54136 | 21.2 | | 19.3 | −9.1 | | 18.6 | −12.6 | |
| | 54490 | 22.3 | | 20.8 | −7.0 | | 19.4 | −13.3 | |
| 2 | 54313 | 21.8 | nothing to report | 21.2 | −2.7 | nothing to report | 20.7 | −5.2 | nothing to report |
| | 54401 | 20.0 | | 18.7 | −6.6 | | 18.4 | −8.1 | |
| | 54478 | 19.4 | | 19.4 | 0.0 | | 18.8 | −2.9 | |
| | 54177 | 21.6 | | 20.9 | −3.1 | | 20.0 | −7.4 | |
| | 54199 | 22.0 | | 20.9 | −5.0 | | 20.2 | −8.1 | |
| | 54102 | 20.8 | | 19.8 | −4.9 | | 19.1 | −8.4 | |
| | 54057 | 22.4 | | 20.7 | −7.6 | | 19.7 | −12.1 | |
| | 54181 | 21.2 | | 20.2 | −4.8 | | 19.1 | −9.9 | |
| 3 | 50398 | 21.4 | mask of pain; | 20.5 | −4.0 | nothing to report | 20.5 | −4.0 | nothing to report |
| | 54079 | 19.9 | slightly rounded | 19.4 | −2.4 | | 19.4 | −2.4 | |
| | 54296 | 20.9 | nose; not ruffled | 20.1 | −4.2 | | 20.1 | −4.2 | |
| | 54341 | 22.6 | hairs; animals | 21.5 | −4.8 | | 21.5 | −4.8 | |
| | 54455 | 23.1 | are active | 21.7 | −6.2 | | 21.7 | −6.2 | |
| | 54235 | 21.2 | | 20.2 | −4.5 | | 20.2 | −4.5 | |
| | 54200 | 21.5 | | 20.9 | −2.8 | | 20.9 | −2.8 | |
| | 57678 | 22.4 | | 21.2 | −5.4 | | 21.2 | −5.4 | |

Group 1: CMP-001 100 μg IV 1 hour D0
Group 2: CMP-001 100 μg IV 30 min D1
Group 3: CMP-001 100 μg IV 15 min D2

### Example 2. Tolerability of CMP-001 IV Bolus in Naive Healthy Mice

A single-administration study in naïve mice was undertaken to investigate the maximum tolerated IV dose of CMP-001 in the absence of anti-Qb antibodies. Chemokine/cytokine response (20-plex Luminex-LMC0006M) at 3 hrs post IV CMP-001 was evaluated.

Naïve mice were administered 0, 100, 300, 600, or 1200 μg CMP-001 (dose amounts in terms of drug substance, G10 CpG oligodeoxynucleotide (SEQ ID NO: 32)) by IV bolus administration.

Mice receiving 0-300 μg CMP-001 showed no clinical signs. Mice receiving 600 CMP-001 were hunched, lethargic, and exhibited piloerection 24 hours post administration. Two of three mice receiving 1200 μg CMP-001 died within one minute, and the third mouse, which due to recoil received 1200 μg CMP-001 in two partial doses administered over 3 min, showed similar clinical signs as the mice receiving 600 μg CMP-001. Clinical signs in mice receiving 600-1200 μg CMP-001 were resolved by 48 hours post-administration.

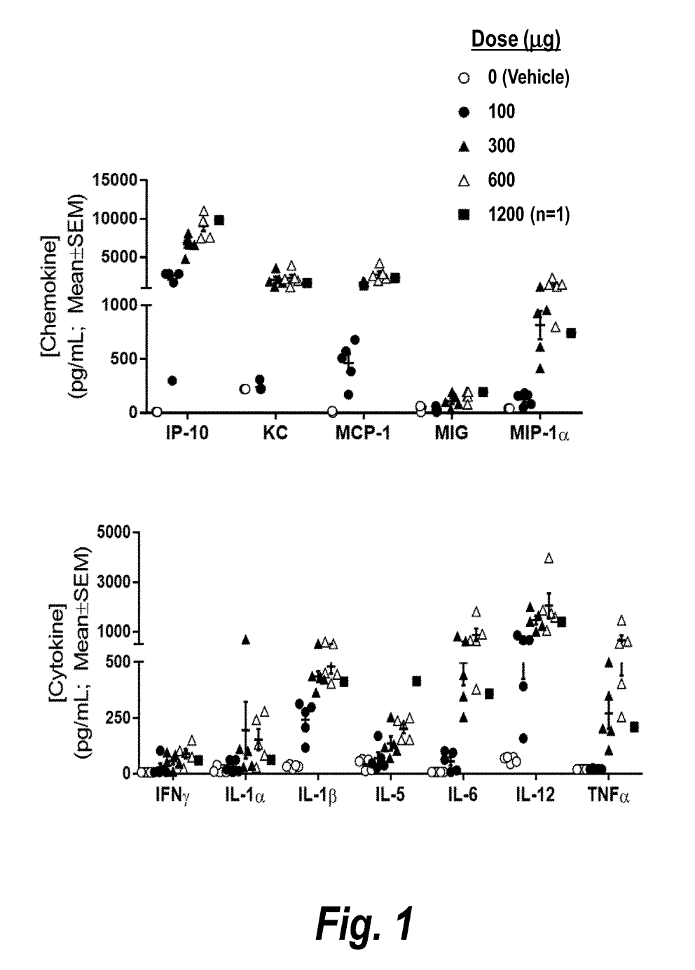

Multiple cytokines and chemokines were upregulated in a dose-dependent manner at 3 hours post-administration of CMP-001 (Table 4 and FIG. 1). Levels of FGF, GM-CSF, IL-2, IL-4, IL-10, IL-13, IL-17, and VEGF were not strongly induced by CMP-001.

TABLE 4
Cytokines, chemokines, and growth factors 3 hours following CMP-001
administration in naïve mice.

| Cytokines | | Chemokines | Growth Factors |
|---|---|---|---|
| GM-CSF (9.8) | IL-6 (8.8) | IP-10 (10) | FGF-basic (24.5) |
| IFN-γ (7.4) | IL-10 (69.6) | KC (221.6) | VEGF (7.6) |
| IL-1α (8.3) | IL-12 p40/P70 (5.4) | MCP-1 (4.6) | |
| IL-1β (16.5) | IL-13 (9.3) | MIG (7.4) | |
| IL-2 (20.1) | IL-17 (1.5) | MIP-1α (41.9) | |
| IL-4 (14.8) | TNF-α (20.7) | | |
| IL-5 (7.2) | | | |

LOD (pg/mL) shown in parentheses.
For FIG. 1, values above used for samples <LOD.

The results from this study demonstrated that the highest well-tolerated IV bolus dose was 600 μg (i.e., 600 μg G10 CpG oligodeoxynucleotide (SEQ ID NO: 32) formulated with 2400 μg Qb (VLP)). The 1200 μg dose showed significant toxicity. The serum analysis at 3 hrs post dose revealed dose-dependent induction of select multiple chemokines and cytokines. See FIG. 1.

### Example 3. Impact of Route of CMP-001 Administration on Biodistribution in CT26 Tumor-Bearing Mice

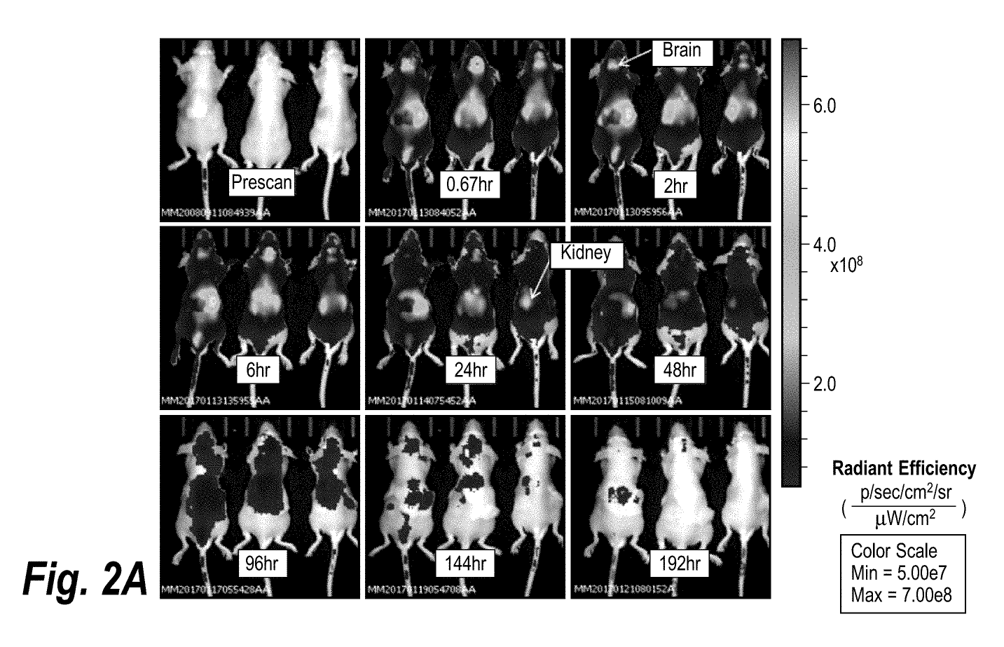

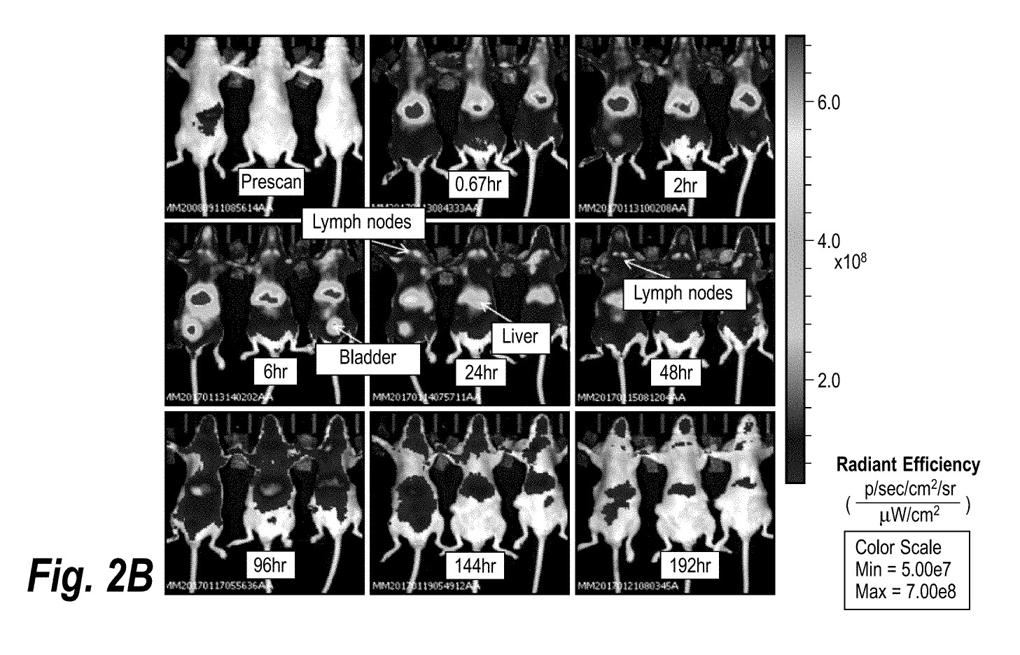

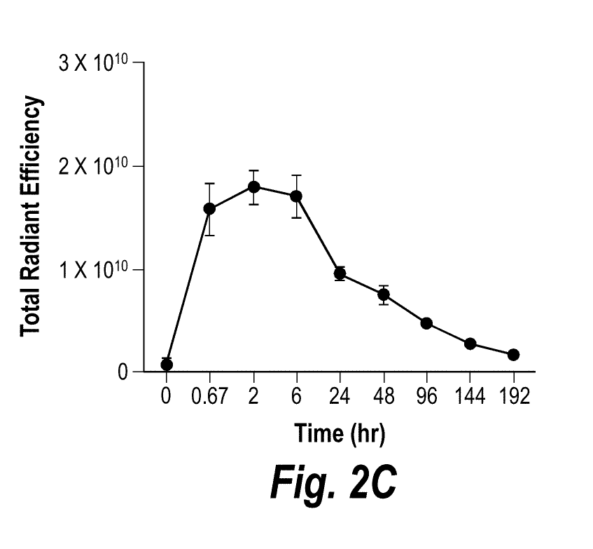

Single doses of fluorescently labeled CMP-001 were administered either intratumorally (IT), subcutaneously (SC), or intravenously (IV). The IT and SC doses were given in both primed and unprimed mice to evaluate the impact of immune complex formation on distribution. The IV dose was given only in the unprimed setting because of toxicity previously observed with IV bolus administration in primed mice. At specified timepoints, biodistribution was imaged using an IVIS kinetics in vivo imaging system (semi-quantitative 2D analysis). Representative results are shown in FIGS. 2A-2C.

TABLE 5
Study design for biodistribution study.

| Grp | N | Prime¹ | Treatment | Route | Dose | Imaging Timepoints |
|---|---|---|---|---|---|---|
| 1 | 3 | Yes | CMP-001- | IT | 100 μg in 20 μL | Pre-scan, 40 min, 2 h, 6 h, |
| | | | Cy5.5 | | | 24 h, 48 h, 96 h, 144 h, 196 h |
| 2 | 3 | No | CMP-001- | IT | 100 μg in 20 μL | Pre-scan, 40 min, 2 h, 6 h, |
| | | | Cy5.5 | | | 24 h, 48 h, 96 h, 144 h, 196 h |
| 3 | 3 | Yes | CMP-001- | SC | 100 μg in 100 μL | Pre-scan, 40 min, 2 h, 6 h, |
| | | | Cy5.5 | | | 24 h, 48 h, 96 h, 144 h, 196 h |
| 4 | 3 | No | CMP-001- | SC | 100 μg in 100 μL | Pre-scan, 40 min, 2 h, 6 h, |
| | | | Cy5.5 | | | 24 h, 48 h, 96 h, 144 h, 196 h |
| 5 | 3 | No | CMP-001- | IV | 100 μg in 100 μL | Pre-scan, 40 min, 2 h, 6 h, |
| | | | Cy5.5 | | | 24 h, 48 h, 96 h, 144 h, 196 h |

¹Prime: 12.5 μg/100 μL CMP-001 SC at D-14

As shown in FIG. 2A, clearance from blood following IV injection appeared to be after 96 hours, and full clearance from the body after 144 hours. As shown in FIG. 2B, retention in liver following IV injection was evident up until 196 hours, and strong signals were evident in mandibular lymph nodes at 24 hours. As shown in FIG. 2C, the liver appeared to be the main organ for accumulation and metabolism of CMP-001-Cy5.5 following IV injection. Peak liver accumulation following IV injection was observed at 2-6 hours, followed by linear clearance kinetics.

### Example 4. IV Administration of CMP-001 in Combination with Anti-PD-1 in Orthotopic Hepa1-6 Mouse Hepatocellular Carcinoma (HCC)

An in vivo efficacy study is underway in an orthotopic model of hepatocellular carcinoma (Hepa1-6). This study is designed to compare the activity of repeat dosing by slow IV infusion vs. single high dose IV bolus. The naked G10 CpG oligonucleotide is also included to compare activity to CMP-001.

TABLE 6
Study design for in vivo HCC efficacy study.

| | | | Anti- | | | | Treatment |
|---|---|---|---|---|---|---|---|
| Grp | N | Prime¹ | PD-1 | Treatment | Dose² | Route | schedule |
| 1 | 10 | No | — | untreated | — | — | — |
| 2 | 10 | Yes | — | Anti-PD-1 alone | 10 mg/kg | IP | TW × 2 |
| 3 | 10 | Yes | No | CMP-001 | 100 μg in 100 μL | IV slow³ | Q5D × 4 |
| 4 | 10 | Yes | Yes | CMP-001 | 100 μg in 100 μL | IV slow³ | Q5D × 4 |
| 5 | 10 | Yes | Yes | G10 | 100 μg in 100 μL | IV | Q5D × 4 |
| 6 | 10 | Yes | Yes | G10 | 300 μg in 100 μL | IV | Q5D × 4 |
| 7 | 10 | No | No | CMP-001 | 200 μg in 100 μL | IV | ×1 |
| 8 | 10 | No | No | CMP-001 | 500 μg in 100 μL | IV | ×1 |
| 9 | 10 | No | Yes | CMP-001 | 200 μg in 100 μL | IV | ×1 |
| 10 | 10 | No | Yes | CMP-001 | 500 μg in 100 μL | IV | ×1 |

¹Prime: 12.5 μg/100 μL CMP-001 SC on D-14.
²Actual CMP-001 dose TBD for groups 7-10 (idea is to test a higher dose range as a single therapeutic dose in unprimed animals).
³Duration of infusion TBD based on results of slow IV infusion tolerability study.

Efficacy is assessed by monitoring serum biomarkers and performing MRI scans. Day 15 and Day 28 serum samples are assayed by ELISA for circulating mouse alpha-fetoprotein (AFP) levels. AFP is a serum biomarker that correlates with tumor burden. MRI analysis on the day following each AFP analysis provides a more detailed assessment of tumor growth in each of the treatment groups. The study activity timeline is as follows.

D-14: injection of primer

D-1 and D0: injection of tumor cells

D5: randomization on body weight and start of treatments

D14: collection of blood and preparation of serum

D15: quantification of AFP in serum samples

D16: MRI

D26: collection of blood and preparation of serum

D27: quantification of AFP in serum samples

D28: MRI

### INCORPORATION BY REFERENCE

All patents and published patent applications mentioned in the description above are incorporated by reference herein in their entirety.

### EQUIVALENTS

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

1. A method of modulating an immune response, comprising
systemically administering to a subject in need thereof an effective amount of a particle comprising a surface and an interior, wherein the surface of the particle comprises an antigen, the interior of the particle comprises an immune modulating agent, and the subject is primed to mount an antibody response to the antigen, to modulate an immune response of the subject.

2-14. (canceled)

15. A method of modulating an immune response, comprising
immunogenically administering to a subject in need thereof an effective amount of a first particle comprising a surface and an interior, wherein the surface of the first particle comprises an antigen, and the interior of the first particle optionally comprises a first immune modulating agent, to immunize the subject against the antigen; and
systemically administering to the subject an effective amount of a second particle comprising a surface and an interior, wherein the surface of the second particle comprises the antigen, and the interior of the second particle comprises a second immune modulating agent, to modulate an immune response of the subject.

16. The method of claim 15, wherein the antigen is selected from the group consisting of viral antigens, bacterial antigens, tumor antigens, and tumor neoantigens.

17. The method of claim 15, wherein the first particle is selected from the group consisting of liposomes, virus-like particles, and lipid nanoparticles.

18. The method of claim 15, wherein the interior of the first particle comprises a first immune modulating agent.

19. (canceled)

20. (canceled)

21. The method of claim 18, wherein the first immune modulating agent is a TLR agonist.

22. The method of claim 21, wherein the first immune modulating agent is a synthetic CpG DNA oligonucleotide.

23. The method of claim 15, wherein the second particle is selected from the group consisting of liposomes, virus-like particles, and lipid nanoparticles.

24. (canceled)

25. The method of claim 15, wherein the second immune modulating agent is selected from the group consisting of therapeutic agents, immune activators, and immune suppressors.

26-28. (canceled)

29. The method of claim 25, wherein the second immune modulating agent is a TLR agonist.

30. The method of claim 29, wherein the second immune modulating agent is a synthetic CpG DNA oligonucleotide.

31. The method of claim 25, wherein the second immune modulating agent is an immune suppressor.

32. The method of claim 31, wherein the immune suppressor is an S-class ODN.

33. (canceled)

34. (canceled)

35. The method of claim 15, wherein the first immune modulating agent and the second immune modulating agent are the same.

36. The method of claim 15, wherein the first immune modulating agent and the second immune modulating agent are different.

37. The method of claim 15, wherein the first particle is administered subcutaneously or intramuscularly.

38. The method of claim 15, wherein the second particle is administered intravenously.

39. The method of claim 15, wherein the subject is a human.

40. A method of treating a disease or condition, comprising
systemically administering to a subject having a disease or condition an effective amount of a particle comprising a surface and an interior, wherein the surface of the particle comprises an antigen, the interior of the particle comprises an immune modulating agent, and the subject is primed to mount an antibody response to the antigen, to modulate an immune response of the subject,
thereby treating the disease or condition.

41-69. (canceled)

70. A method of treating a disease or condition, comprising
immunogenically administering to a subject having a disease or condition an effective amount of a first particle comprising a surface and an interior, wherein the surface of the first particle comprises an antigen, and the interior of the first particle optionally comprises a first immune modulating agent, to immunize the subject against the antigen; and
systemically administering to the subject an effective amount of a second particle comprising a surface and an interior, wherein the surface of the second particle comprises the antigen, and the interior of the second particle comprises a second immune modulating agent, to modulate an immune response of the subject,
thereby treating the disease or condition.

71-110. (canceled)
